SEQUENCE LISTING (18 sequences)

SEQ ID NO: 1 - protein, 461 aa, synthetic construct
MKTLQFFFLFCCWKAICCNSCELTNITIAIEKEECRFCISINTTWCAGYCYTRDLVYKDPARPKIQKTCTFKELVYETVRVPGCAHHADSLYTYPVATQCHCGKCDSDSTDCTVRGLGPSYCSFGEMKEGGGSGGGSGGGSGGGAPDVQDCPECTLQENPFFSQPGAPILQCMGCCFSRAYPTPLRSKKTMLVQKNVTSESTCCVAKSYNRVTVMGGFKVENHTACHCSTCYYHKSASTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 2 - DNA, 1392 bp, synthetic construct
  source 1..1392: mol_type = other DNA
gccaccatgaagaccctgcagttcttcttcctgttctgctgctggaaggccatctgctgcaacagctgcgagctgaccaacatcacaatcgccatcgagaaagaggaatgccggttctgcatcagcatcaacaccacttggtgcgccggctactgctacacccgggacctggtgtacaaggaccccgccagacccaagatccagaaaacctgcaccttcaaagaactggtgtacgagacagtgcgggtgcccggatgtgcccaccatgccgatagcctgtacacctaccctgtggccacccagtgccactgcggcaagtgcgatagcgacagcaccgattgcaccgtgcggggactgggccctagctactgtagcttcggcgagatgaaggaaggcggcggatctggcggaggaagcggagggggatctgggggcggagcacctgatgtgcaggattgccctgagtgcaccctgcaggaaaacccattcttcagccagcctggcgcccctatcctgcagtgcatgggctgctgcttcagcagagcctaccccacccccctgcggagcaagaaaaccatgctggtgcagaaaaacgtgaccagcgagagcacctgttgcgtggccaagagctacaacagagtgaccgtgatgggcggcttcaaggtggaaaaccacaccgcctgccactgcagcacatgctactaccacaagagcgctagcaccaccacccctgcccctagacctccaacacccgcccctacaatcgcctcccagcctctgtctctgaggcccgaggcttgtagaccagctgctggcggagccgtgcacaccagaggactggatttcgcctgcgacatctacatctgggcccctctggccggcacatgtggcgtgctgctgctgagcctcgtgatcaccctgtactgcaagcggggcagaaagaagctgctgtacatcttcaagcagcccttcatgcggcccgtgcagaccacccaggaagaggacggctgctcctgcagattccccgaagaggaagaggggggctgcgaactgagagtgaagttcagcagaagcgccgacgcccctgcctacaagcagggccagaaccagctgtacaacgagctgaacctgggcagacgggaagagtacgacgtgctggacaagcggagaggcagggaccctgagatgggcggaaagcccagacggaagaacccccaggaaggcctgtataacgaactgcagaaagacaagatggccgaggcctacagcgagatcggaatgaagggcgagcggagaagaggcaagggccacgatggcctgtaccagggcctgagcaccgccaccaaggacacctatgacgccctgcacatgcaggccctgccccccagataa

SEQ ID NO: 3 - RNA, 36 nt, synthetic construct
  source 1..36: mol_type = other RNA
tgcacgtagtctgagtgctggttttagagctagaaa

SEQ ID NO: 4 - DNA, 23 bp, synthetic construct
  source 1..23: mol_type = other DNA
ctgaccaacatcacaatcgccat

SEQ ID NO: 5 - DNA, 23 bp, synthetic construct
  source 1..23: mol_type = other DNA
gttttctggatcttgggtctggc

SEQ ID NO: 6 - DNA, 20 bp, synthetic construct
  source 1..20: mol_type = other DNA
acagagtgaccgtgatgggc

SEQ ID NO: 7 - DNA, 20 bp, synthetic construct
  source 1..20: mol_type = other DNA
ctgggaggcgattgtagggg

SEQ ID NO: 8 - DNA, 300 bp, synthetic construct
  source 1..300: mol_type = other DNA
cacgcacctgagccccgaggagaaggcgctgaggaggaaactgaaaaacagagtagcagctcagactgccagagatcgaaagaaggctcgaatgagtgagctggaacagcaagtggtagatttagaagaagagaaccaaaaacttttgctagaaaatcagcttttacgagagaaaactcatggccttgtagttgagaaccaggagttaagacagcgcttggggatggatgccctggttgctgaagaggaggcggaagccaaggggaatgaagtgaggccagtggccgggtctgctgagtc

SEQ ID NO: 9 - DNA, 59 bp, synthetic construct
  source 1..59: mol_type = other DNA
tggccacgaacttctccctgttaaagcaagcaggagacgtggaagaaaaccccggtccc

SEQ ID NO: 10 - DNA, 1386 bp, synthetic construct
  source 1..1386: mol_type = other DNA
atgaagaccctgcagttcttcttcctgttctgctgctggaaggccatctgctgcaacagctgcgagctgaccaacatcacaatcgccatcgagaaagaggaatgccggttctgcatcagcatcaacaccacttggtgcgccggctactgctacacccgggacctggtgtacaaggaccccgccagacccaagatccagaaaacctgcaccttcaaagaactggtgtacgagacagtgcgggtgcccggatgtgcccaccatgccgatagcctgtacacctaccctgtggccacccagtgtcactgcggcaagtgcgatagcgacagcaccgattgcaccgtgcggggactgggccctagctactgtagcttcggcgagatgaaggaaggcggcggatctggcggaggaagcggagggggatctgggggcggagcacctgatgtgcaggattgccctgagtgcaccctgcaggaaaacccattcttcagccagcctggcgcccctatcctgcagtgcatgggctgctgcttcagcagagcctaccccacccccctgcggagcaagaaaaccatgctggtgcagaaaaacgtgaccagcgagagcacctgttgcgtggccaagagctacaacagagtgaccgtgatgggcggcttcaaggtggaaaaccacaccgcctgccactgcagcacatgctactaccacaagagcgctagcaccaccacccctgcccctagacctccaacacccgcccctacaatcgcctcccagcctctgtctctgaggcccgaggcttgtagaccagctgctggcggagccgtgcacaccagaggactggatttcgcctgcgacatctacatctgggcccctctggccggcacatgtggcgtgctgctgctgagcctcgtgatcaccctgtactgcaagcggggcagaaagaagctgctgtacatcttcaagcagcccttcatgcggcccgtgcagaccacccaggaagaggacggctgctcctgcagattccccgaagaggaagaggggggctgcgaactgagagtgaagttcagcagaagcgccgacgcccctgcctacaagcagggccagaaccagctgtacaacgagctgaacctgggcagacgggaagagtacgacgtgctggacaagcggagaggcagggaccctgagatgggcggaaagcccagacggaagaacccccaggaaggcctgtataacgaactgcagaaagacaagatggccgaggcctacagcgagatcggaatgaagggcgagcggagaagaggcaagggccacgatggcctgtaccagggcctgagcaccgccaccaaggacacctatgacgccctgcacatgcaggccctgccccccagataa

SEQ ID NO: 11 - DNA, 301 bp, synthetic construct
  source 1..301: mol_type = other DNA
cgcagcactcagactacgtgcacctctgcagcaggtgcaggcccagttgtcacccctccagaacatctccccatggattctggcggtattgactcttcagattcagagtctgatatcctgttgggcattctggacaacttggacccagtcatgttcttcaaatgcccttccccagagcctgccagcctggaggagctcccagaggtctacccagaaggacccagttccttaccagcctccctttctctgtcagtggggacgtcatcagccaagctggaagccattaatgaactaattcgtt

SEQ ID NO: 12 - DNA, 2046 bp, synthetic construct
  source 1..2046: mol_type = other DNA
cacgcacctgagccccgaggagaaggcgctgaggaggaaactgaaaaacagagtagcagctcagactgccagagatcgaaagaaggctcgaatgagtgagctggaacagcaagtggtagatttagaagaagagaaccaaaaacttttgctagaaaatcagcttttacgagagaaaactcatggccttgtagttgagaaccaggagttaagacagcgcttggggatggatgccctggttgctgaagaggaggcggaagccaaggggaatgaagtgaggccagtggccgggtctgctgagtctggccacgaacttctccctgttaaagcaagcaggagacgtggaagaaaaccccggtcccatgaagaccctgcagttcttcttcctgttctgctgctggaaggccatctgctgcaacagctgcgagctgaccaacatcacaatcgccatcgagaaagaggaatgccggttctgcatcagcatcaacaccacttggtgcgccggctactgctacacccgggacctggtgtacaaggaccccgccagacccaagatccagaaaacctgcaccttcaaagaactggtgtacgagacagtgcgggtgcccggatgtgcccaccatgccgatagcctgtacacctaccctgtggccacccagtgtcactgcggcaagtgcgatagcgacagcaccgattgcaccgtgcggggactgggccctagctactgtagcttcggcgagatgaaggaaggcggcggatctggcggaggaagcggagggggatctgggggcggagcacctgatgtgcaggattgccctgagtgcaccctgcaggaaaacccattcttcagccagcctggcgcccctatcctgcagtgcatgggctgctgcttcagcagagcctaccccacccccctgcggagcaagaaaaccatgctggtgcagaaaaacgtgaccagcgagagcacctgttgcgtggccaagagctacaacagagtgaccgtgatgggcggcttcaaggtggaaaaccacaccgcctgccactgcagcacatgctactaccacaagagcgctagcaccaccacccctgcccctagacctccaacacccgcccctacaatcgcctcccagcctctgtctctgaggcccgaggcttgtagaccagctgctggcggagccgtgcacaccagaggactggatttcgcctgcgacatctacatctgggcccctctggccggcacatgtggcgtgctgctgctgagcctcgtgatcaccctgtactgcaagcggggcagaaagaagctgctgtacatcttcaagcagcccttcatgcggcccgtgcagaccacccaggaagaggacggctgctcctgcagattccccgaagaggaagaggggggctgcgaactgagagtgaagttcagcagaagcgccgacgcccctgcctacaagcagggccagaaccagctgtacaacgagctgaacctgggcagacgggaagagtacgacgtgctggacaagcggagaggcagggaccctgagatgggcggaaagcccagacggaagaacccccaggaaggcctgtataacgaactgcagaaagacaagatggccgaggcctacagcgagatcggaatgaagggcgagcggagaagaggcaagggccacgatggcctgtaccagggcctgagcaccgccaccaaggacacctatgacgccctgcacatgcaggccctgccccccagataacgcagcactcagactacgtgcacctctgcagcaggtgcaggcccagttgtcacccctccagaacatctccccatggattctggcggtattgactcttcagattcagagtctgatatcctgttgggcattctggacaacttggacccagtcatgttcttcaaatgcccttccccagagcctgccagcctggaggagctcccagaggtctacccagaaggacccagttccttaccagcctccctttctctgtcagtggggacgtcatcagccaagctggaagccattaatgaactaattcgtt

SEQ ID NO: 13 - DNA, 300 bp, synthetic construct
  source 1..300: mol_type = other DNA
cagcactttagatgggagaattgctgccttggatcctgaaaatcatggtaaaaagcagtgggatttggatgtgggatccggttccttggtgtcatccagccttagcaaaccagaggtatttgggaataagatgatcattccttccctggatggagccctcttccagtgggaccaagaccgtgaaagcatggaaacagttcctttcacagttgaatcacttcttgaatcttcttataaatttggagatgatgttgttttggttggaggaaaatctctgactacatatggactcagtgcata

SEQ ID NO: 14 - DNA, 59 bp, synthetic construct; identical to SEQ ID NO: 9
  source 1..59: mol_type = other DNA

SEQ ID NO: 15 - DNA, 299 bp, synthetic construct
  source 1..299: mol_type = other DNA
tagtggaaaggtgagatggactcagtgcatatagtggaaaggtaagtgaaaatgctgaatttactttggggaaatcagagtaaattagggtagaaaaagtaatttattaaactacacttattattagttgagttttattgtaattttcccctgaggttgtcatttgttttaataagagaactgtgaggtaggaaggggaaactaataacagaataaatggcagagccaggaatagcaggaggaagagaattcataaatatggtctactgtgtctcaggtgttttttttttttttttttt

SEQ ID NO: 16 - DNA, 2044 bp, synthetic construct
  source 1..2044: mol_type = other DNA
cagcactttagatgggagaattgctgccttggatcctgaaaatcatggtaaaaagcagtgggatttggatgtgggatccggttccttggtgtcatccagccttagcaaaccagaggtatttgggaataagatgatcattccttccctggatggagccctcttccagtgggaccaagaccgtgaaagcatggaaacagttcctttcacagttgaatcacttcttgaatcttcttataaatttggagatgatgttgttttggttggaggaaaatctctgactacatatggactcagtgcatatggccacgaacttctccctgttaaagcaagcaggagacgtggaagaaaaccccggtcccatgaagaccctgcagttcttcttcctgttctgctgctggaaggccatctgctgcaacagctgcgagctgaccaacatcacaatcgccatcgagaaagaggaatgccggttctgcatcagcatcaacaccacttggtgcgccggctactgctacacccgggacctggtgtacaaggaccccgccagacccaagatccagaaaacctgcaccttcaaagaactggtgtacgagacagtgcgggtgcccggatgtgcccaccatgccgatagcctgtacacctaccctgtggccacccagtgtcactgcggcaagtgcgatagcgacagcaccgattgcaccgtgcggggactgggccctagctactgtagcttcggcgagatgaaggaaggcggcggatctggcggaggaagcggagggggatctgggggcggagcacctgatgtgcaggattgccctgagtgcaccctgcaggaaaacccattcttcagccagcctggcgcccctatcctgcagtgcatgggctgctgcttcagcagagcctaccccacccccctgcggagcaagaaaaccatgctggtgcagaaaaacgtgaccagcgagagcacctgttgcgtggccaagagctacaacagagtgaccgtgatgggcggcttcaaggtggaaaaccacaccgcctgccactgcagcacatgctactaccacaagagcgctagcaccaccacccctgcccctagacctccaacacccgcccctacaatcgcctcccagcctctgtctctgaggcccgaggcttgtagaccagctgctggcggagccgtgcacaccagaggactggatttcgcctgcgacatctacatctgggcccctctggccggcacatgtggcgtgctgctgctgagcctcgtgatcaccctgtactgcaagcggggcagaaagaagctgctgtacatcttcaagcagcccttcatgcggcccgtgcagaccacccaggaagaggacggctgctcctgcagattccccgaagaggaagaggggggctgcgaactgagagtgaagttcagcagaagcgccgacgcccctgcctacaagcagggccagaaccagctgtacaacgagctgaacctgggcagacgggaagagtacgacgtgctggacaagcggagaggcagggaccctgagatgggcggaaagcccagacggaagaacccccaggaaggcctgtataacgaactgcagaaagacaagatggccgaggcctacagcgagatcggaatgaagggcgagcggagaagaggcaagggccacgatggcctgtaccagggcctgagcaccgccaccaaggacacctatgacgccctgcacatgcaggccctgccccccagataatagtggaaaggtgagatggactcagtgcatatagtggaaaggtaagtgaaaatgctgaatttactttggggaaatcagagtaaattagggtagaaaaagtaatttattaaactacacttattattagttgagttttattgtaattttcccctgaggttgtcatttgttttaataagagaactgtgaggtaggaaggggaaactaataacagaataaatggcagagccaggaatagcaggaggaagagaattcataaatatggtctactgtgtctcaggtgttttttttttttttttttt

SEQ ID NO: 17 - protein, 11 aa, synthetic construct
HQQIDDFLCEV

SEQ ID NO: 18 - RNA, 36 nt, synthetic construct
  source 1..36: mol_type = other RNA
tatggactcagtgcatataggttttagagctagaaa

(19) United States
(12) Patent Application Publication
(10) Pub. No.: US 2026/0335364 A1
(43) Pub. Date: Oct. 29, 2026

(54) CAR-T CELLS WITH ABLATED ER SRESS

(71) Applicant: H. LEE MOFFITT CANCER CENTER AND RESEARCH INSTITUTE INC., Tampa, FL (US)

(72) Inventors: Jose CONEJO-GARCIA, Durham, NC (US); Paulo RODRIGUEZ, Tampa, FL (US)

(21) Appl. No.: 19/109,779

(22) PCT Filed: Sep. 14, 2023
(86) PCT No.: PCT/US2023/074192

§ 371 Date: Mar. 7, 2025

Related U.S. Application Data
(60) Provisional application No. 63/375,628, filed on Sep. 14, 2022.

(51) Int. Cl. A61K 35/17; A61K 40/11; A61K 40/31; A61K 40/32; A61K 40/42; C07K 14/59; C07K 14/705; C07K 16/28; C12N 5/0783; C12N 15/11

(52) U.S. Cl. CPC A61K 35/17; A61K 40/11; A61K 40/31; A61K 40/32; A61K 40/4237; C07K 14/59; C07K 14/7051; C07K 16/2818; C07K 16/2827; C12N 5/0636; C12N 15/11; A61K 2239/59; C07K 2319/33

(57) ABSTRACT

Disclosed herein are non-viral methods to ablate Activating transcription factor 6 (ATF6), X-box binding protein 1 (XBP1), and/or protein kinase R (PKR)-like endoplasmic reticulum kinase (PERK) in T cells while effectively expressing chimeric receptors. Therefore, disclosed herein is a chimeric cell expressing a chimeric receptor, wherein the chimeric receptor is encoded by a transgene, and wherein the transgene is inserted in the genome of the cell at a location that disrupts expression or activity of an endogenous ATF6, XBP1, and/or PERK protein.

### CROSS-REFERENCE TO RELATED APPLICATIONS

This application claims benefit of U.S. Provisional Application No. 63/375,628, filed Sep. 14, 2022, which is hereby incorporated herein by reference in its entirety.

### SEQUENCE LISTING

This application contains a sequence listing filed in ST.26 format entitled “320803_2930_Sequence_Listing” created on Sep. 8, 2023, and having 25, 168 bytes. The content of the sequence listing is incorporated herein in its entirety.

### BACKGROUND

In recent years, transferring autologous T cells engineered to express chimeric antigen receptors (CARs) has shown impressive cures for patients with hematologic malignancies (Porter, D. L., et al. N Engl J Med 2011 365:725-733; Maus, M. V., et al. Blood 2014 123:2625-2635; Kalos, M., et al. Sci Transl Med 2011 3: 95ra73). Several hurdles, however, have so far prevented the success of CAR T cells against solid tumors, including ovarian cancer.

### SUMMARY

Therefore, disclosed herein is a way to circumvent the use of viral vectors to create CAR-T cells, while ablating intrinsic drivers of their paralysis, both of which could enable better therapies and reduce the cost of cell therapy.

In particular, disclosed herein are non-viral methods to ablate the effects of endoplasmic reticulum (ER) stress by genetically editing Activating transcription factor 6 (ATF6), X-box binding protein 1 (XBP1), and/or protein kinase R (PKR)-like endoplasmic reticulum kinase (PERK) in T cells while effectively expressing chimeric receptors. In particular, disclosed herein is a T cell expressing a chimeric receptor, wherein the chimeric receptor is encoded by a transgene, and wherein the transgene is inserted in the genome of the cell at a location that disrupts expression or activity of endogenous ATF6, XBP1, and/or PERK.

In some embodiments, the transgene is inserted into an ATF6, XBP1, and/or PERK gene loci, thereby disrupting gene transcription and ensuring constitutive expression of the transgene. The transgene can be inserted at any loci within the ATF6, XBP1, and/or PERK gene that would disrupt gene transcription while also allowing for transcription of the transgene.

For example, in some cases, the transgene is inserted into exon 4 of XBP1. For example, in some cases, the transgene is inserted into exon 3 of EIF2AK3.1 (NM_004836.7, corresponding to exon 1 of EIF2AK3.2: NM_001313915.2).

Site-specific insertion of the transgene can be done, for example, by gene editing techniques, such as CRISPR or TALEN. In some cases, 2 different gene editing systems are used: one for integration of the transgene, and another one for effective ablation of all FoxP1 variants (for instance, with a target at exon 10, common to all of them).

In some embodiments, the chimeric receptor comprises a chimeric antigen receptor (CAR) polypeptide. CARs generally combine an antigen recognition domain with transmembrane signaling motifs involved in lymphocyte activation. The antigen recognition domain can be, for example, the single-chain variable fragments (scFv) of a monoclonal antibody (mAb) or a fragment of a natural ligand that binds a target receptor. CARs are generally made up of three domains: an ectodomain, a transmembrane domain, and an endodomain. The ectodomain comprises the antigen recognition domain. It also optionally contains a signal peptide (SP) so that the CAR can be glycosylated and anchored in the cell membrane of the immune effector cell. The transmembrane domain (TD), is as its name suggests, connects the ectodomain to the endodomain and resides within the cell membrane when expressed by a cell. The endodomain is the business end of the CAR that transmits an activation signal to the immune effector cell after antigen recognition. For example, the endodomain can contain an intracellular signaling domain (ISD) and optionally a co-stimulatory signaling region (CSR).

In some embodiments, the chimeric receptor comprises two subunits of a follicule-stimulating hormone (FSH), which binds to FSH receptors. These chimeric receptors are referenced to herein as “chimeric endocrine receptors (CERs)” and can target FSH-positive ovarian tumors. In some embodiments, the CER contains two subunits of FSH (FSHB and CGa), separated by a linker.

In some embodiments, the chimeric receptor has the following structure:

Signal Peptide (FSH beta)_hFSH beta_Spacer_hFSH alpha_Hinge (from human CD8)_TM domain (from human CD8)_human 4-1BB (intracellular)_human CD3z domain.

In some embodiments, the chimeric receptor has the amino acid sequence: MKTLQFFFLFCCWKAICC_NSCELTNITIAIEKEECRFCISINTTWCAGYCYTRDLVYKD PARPKIQKTCTFKELVYETVRVPGCAHHADSLYTYPVATQCHCGKCDSDSTDCTVRG LGPSYCSFGEMKE_GGGSGGGSGGGSGGG_APDVQDCPECTLQENPFFSQPGAPIL QCMGCCFSRAYPTPLRSKKTMLVQKNVTSESTCCVAKSYNRVTVMGGFKVENHTAC HCSTCYYHK_SASTTTPAPRPPTPAPTIASQPLSLRPEACRPAAGGAVHTRGLDFACD_I YIWAPLAGTCGVLLLSLVITLYC_KRGRKKLLYIFKQPFMRPVQTTQEEDGCSCRFPEE EEGGCEL_RVKFSRSADAPAYKQGQNQLYNELNLGRREEYDVLDKRRGRDPEMGGK PRRKNPQEGLYNELQKDKMAEAYSEIGMKGERRRGKGHDGLYQGLSTATKDTYDALH MQALPPR (SEQ ID NO:1), or a variant thereof having at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% sequence identity to SEQ ID NO:1.

Therefore, in some embodiments, the chimeric receptor is encoded by the following nucleic acid sequence:

(SEQ ID NO: 2)
GCCACCATGAAGACCCTGCAGTTCTTCTTCCTGTTCTGCTGCTGGAAGGC
CATCTGCTGCAACAGCTGCGAGCTGACCAACATCACAATCGCCATCGAGA
AAGAGGAATGCCGGTTCTGCATCAGCATCAACACCACTTGGTGCGCCGGC
TACTGCTACACCCGGGACCTGGTGTACAAGGACCCCGCCAGACCCAAGAT
CCAGAAAACCTGCACCTTCAAAGAACTGGTGTACGAGACAGTGCGGGTGC
CCGGATGTGCCCACCATGCCGATAGCCTGTACACCTACCCTGTGGCCACC
CAGTGCCACTGCGGCAAGTGCGATAGCGACAGCACCGATTGCACCGTGCG
GGGACTGGGCCCTAGCTACTGTAGCTTCGGCGAGATGAAGGAAGGCGGCG
GATCTGGCGGAGGAAGCGGAGGGGGATCTGGGGGGGGAGCACCTGATGTG
CAGGATTGCCCTGAGTGCACCCTGCAGGAAAACCCATTCTTCAGCCAGCC
TGGCGCCCCTATCCTGCAGTGCATGGGCTGCTGCTTCAGCAGAGCCTACC
CCACCCCCCTGCGGAGCAAGAAAACCATGCTGGTGCAGAAAAACGTGACC
AGCGAGAGCACCTGTTGCGTGGCCAAGAGCTACAACAGAGTGACCGTGAT
GGGCGGCTTCAAGGTGGAAAACCACACCGCCTGCCACTGCAGCACATGCT
ACTACCACAAGAGCGCTAGCACCACCACCCCTGCCCCTAGACCTCCAACA
CCCGCCCCTACAATCGCCTCCCAGCCTCTGTCTCTGAGGCCCGAGGCTTG
TAGACCAGCTGCTGGCGGAGCCGTGCACACCAGAGGACTGGATTTCGCCT
GCGACATCTACATCTGGGCCCCTCTGGCCGGCACATGTGGCGTGCTGCTG
CTGAGCCTCGTGATCACCCTGTACTGCAAGCGGGGCAGAAAGAAGCTGCT
GTACATCTTCAAGCAGCCCTTCATGCGGCCCGTGCAGACCACCCAGGAAG
AGGACGGCTGCTCCTGCAGATTCCCCGAAGAGGAAGAGGGGGGCTGCGAA
CTGAGAGTGAAGTTCAGCAGAAGCGCCGACGCCCCTGCCTACAAGCAGGG
CCAGAACCAGCTGTACAACGAGCTGAACCTGGGCAGACGGGAAGAGTACG
ACGTGCTGGACAAGCGGAGAGGCAGGGACCCTGAGATGGGCGGAAAGCCC
AGACGGAAGAACCCCCAGGAAGGCCTGTATAACGAACTGCAGAAAGACAA
GATGGCCGAGGCCTACAGCGAGATCGGAATGAAGGGCGAGCGGAGAAGAG
GCAAGGGCCACGATGGCCTGTACCAGGGCCTGAGCACCGCCACCAAGGAC
ACCTATGACGCCCTGCACATGCAGGCCCTGCCCCCCAGATAA.

Also disclosed are isolated nucleic acid sequences encoding the disclosed polypeptides, vectors comprising these isolated nucleic acids, and cells containing these vectors. The chimeric cells disclosed herein can be an immune effector cell selected from the group consisting of an alpha-beta T cells, a gamma-delta T cell, a Natural Killer (NK) cells, a Natural Killer T (NKT) cell, a B cell, an innate lymphoid cell (ILC), a cytokine induced killer (CIK) cell, a cytotoxic T lymphocyte (CTL), a lymphokine activated killer (LAK) cell, and a regulatory T cell. In some embodiments, the cell exhibits an anti-tumor immunity when the antigen binding domain of the CAR binds to CD123.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIGS. 1A and 1B show an FSH-CER HDR construct used to replace XBP1 and express FSHCER. FIG. 1A shows a 1.3 kb DNA sequence encoding FSH-CER is spanned by 2 arms of approximately 300 bp, to drive XBP1 elimination. FIG. 1B shows CRISPR-based deletion of XBP1 (left; clone #EPR22004) and expression of FSH (right; hCGa clone #381012) in primary human T cells by FACS analysis.

FIG. 2 illustrates an FSH-CER HDR construct used to replace PERK and express FSHCER.

FIGS. 3A to 3C show functional integration of an OR2H1-targeted, Vy8V01 “HIT” construct into the PERK locus. FIGS. 3A and 3B illustrate generation of AAV6 expressing the VH sequence of the targeting domain of OR2H1 CAR1 (Martin, A.L., et al. Mol Cancer Ther. 2022 21 (7): 1184-1194), fused to the TCR Cy8 domain, plus the CAR VL sequence fused to the Cδ1 sequence, resulting in chimeric VHCγ8 and VLCδ1 chains linked by a cleavage inducing P2A element, plus a polyA sequence. This construct was spanned by 300 bp arms homologous to the sequence at the 5′- an 3′-ends of the cutting site of a CRISPR guide designed for the indicated site, to serve as HDR templates. Using a Neon protocol (Roth, T. L., et al. Nature 2018 559:405-409; Osborn, M. J., et al. Mol Ther 2016 24:570-581; Rahdar, M., et al. Proc Natl Acad Sci USA 2015 112: E7110-7117). CAS9-based ctRNP complexes with CAS9 plus a tracRNA oligo and a crRNA oligo with the PERK-specific guide for the indicated position were co-electroporated. Recombinant AAV6 donor vectors were added 1 h after electroporation and edited cells were cultured using standard conditions. This system results in ablation of PERK and redirection of T cell specificity towards OR2H1+tumor cells, to trigger Vδ1 TCR activation and corresponding cytotoxicity. To validate recognition of OR2H1 by T cells expressing “HIT” constructs integrated into the PERK locus we tetramerized the (biotinylated) antigen specifically recognized by the scFv of the OR2H1 CAR (HQQIDDFLCEV, SEQ ID NO:17) with APC-streptavidin. FIG. 3C shows 53% of the T cells recognized the antigen, compared to T cells without AAV incubation.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in °C, and pressure is at or near atmospheric. Standard temperature and pressure are defined as 20 °C and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise.

The term “subject” refers to any individual who is the target of administration or treatment. The subject can be a vertebrate, for example, a mammal. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

The term “sample from a subject” refers to a tissue (e.g., tissue biopsy), organ, cell (including a cell maintained in culture), cell lysate (or lysate fraction), biomolecule derived from a cell or cellular material (e.g. a polypeptide or nucleic acid), or body fluid from a subject. Non-limiting examples of body fluids include blood, urine, plasma, serum, tears, lymph, bile, cerebrospinal fluid, interstitial fluid, aqueous or vitreous humor, colostrum, sputum, amniotic fluid, saliva, anal and vaginal secretions, perspiration, semen, transudate, exudate, and synovial fluid.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

ATF6, XBP1, and/or PERK Disruption

Disclosed herein are non-viral methods to ablate ATF6, XBP1, and/or PERK in T cells while effectively expressing chimeric receptors. Therefore, disclosed herein is a chimeric cell expressing a chimeric receptor, wherein the chimeric receptor is encoded by a transgene, and wherein the transgene is inserted in the genome of the cell at a location that disrupts expression or activity of an endogenous ATF6, XBP1, and/or PERK protein.

In some embodiments, the transgene is inserted into the ATF6, XBP1, and/or PERK gene loci, thereby disrupting gene transcription. The transgene can be inserted at any loci within the ATF6, XBP1, and/or PERK gene that would disrupt gene transcription.

Site-specific insertion of the transgene can be done, for example, by gene editing techniques, such as CRISPR.

### Chimeric Endocrine Receptors (CERs)

In some embodiments, the chimeric receptor comprises two subunits of a follicule-stimulating hormone (FSH), which binds FSH receptors. These chimeric receptors are referenced to herein as “chimeric endocrine receptors (CERs)” and can target FSH-positive ovarian tumors. In some embodiments, the CER contains two subunits of FSH (FSHB and CGa), separated by a linker.

### Chimeric Antigen Receptors (CAR)

CARs generally incorporate an antigen recognition domain from the single-chain variable fragments (scFv) of a monoclonal antibody (mAb) with transmembrane signaling motifs involved in lymphocyte activation (Sadelain M, et al. Nat Rev Cancer 2003 3:35-45). The disclosed CAR is generally made up of three domains: an ectodomain, a transmembrane domain, and an endodomain. The ectodomain comprises the recognition domain. It also optionally contains a signal peptide (SP) so that the CAR can be glycosylated and anchored in the cell membrane of the immune effector cell. The transmembrane domain (TD), is as its name suggests, connects the ectodomain to the endodomain and resides within the cell membrane when expressed by a cell. The endodomain is the business end of the CAR that transmits an activation signal to the immune effector cell after antigen recognition. For example, the endodomain can contain an intracellular signaling domain (ISD) and optionally a co-stimulatory signaling region (CSR).

A “signaling domain (SD)” generally contains immunoreceptor tyrosine-based activation motifs (ITAMs) that activate a signaling cascade when the ITAM is phosphorylated. The term “co-stimulatory signaling region (CSR)” refers to intracellular signaling domains from costimulatory protein receptors, such as CD28, 41BB, and ICOS, that are able to enhance T-cell activation by T-cell receptors.

In some embodiments, the endodomain contains an SD or a CSR, but not both. In these embodiments, an immune effector cell containing the disclosed CAR is only activated if another CAR (or a T-cell receptor) containing the missing domain also binds its respective antigen.

Additional CAR constructs are described, for example, in Fresnak A D, et al. Engineered T cells: the promise and challenges of cancer immunotherapy. Nat Rev Cancer. 2016 Aug. 23; 16 (9): 566-81, which is incorporated by reference in its entirety for the teaching of these CAR models.

For example, the CAR can be a TRUCK, Universal CAR, Self-driving CAR, Armored CAR, Self-destruct CAR, Conditional CAR, Marked CAR, TenCAR, Dual CAR, or sCAR.

TRUCKs (T cells redirected for universal cytokine killing) co-express a chimeric antigen receptor (CAR) and an antitumor cytokine. Cytokine expression may be constitutive or induced by T cell activation. Targeted by CAR specificity, localized production of pro-inflammatory cytokines recruits endogenous immune cells to tumor sites and may potentiate an antitumor response.

Universal, allogeneic CAR T cells are engineered to no longer express endogenous T cell receptor (TCR) and/or major histocompatibility complex (MHC) molecules, thereby preventing graft-versus-host disease (GVHD) or rejection, respectively.

Self-driving CARs co-express a CAR and a chemokine receptor, which binds to a tumor ligand, thereby enhancing tumor homing.

CAR T cells engineered to be resistant to immunosuppression (Armored CARs) may be genetically modified to no longer express various immune checkpoint molecules (for example, cytotoxic T lymphocyte-associated antigen 4 (CTLA4) or programmed cell death protein 1 (PD1)), with an immune checkpoint switch receptor, or may be administered with a monoclonal antibody that blocks immune checkpoint signaling.

A self-destruct CAR may be designed using RNA delivered by electroporation to encode the CAR. Alternatively, inducible apoptosis of the T cell may be achieved based on ganciclovir binding to thymidine kinase in gene-modified lymphocytes or the more recently described system of activation of human caspase 9 by a small-molecule dimerizer.

A conditional CAR T cell is by default unresponsive, or switched ‘off’, until the addition of a small molecule to complete the circuit, enabling full transduction of both signal 1 and signal 2, thereby activating the CAR T cell. Alternatively, T cells may be engineered to express an adaptor-specific receptor with affinity for subsequently administered secondary antibodies directed at target antigen.

Marked CAR T cells express a CAR plus a tumor epitope to which an existing monoclonal antibody agent binds. In the setting of intolerable adverse effects, administration of the monoclonal antibody clears the CAR T cells and alleviates symptoms with no additional off-tumor effects.

A tandem CAR (TanCAR) T cell expresses a single CAR consisting of two linked single-chain variable fragments (scFvs) that have different affinities fused to intracellular co-stimulatory domain(s) and a CD33 domain. TanCAR T cell activation is achieved only when target cells co-express both targets.

A dual CAR T cell expresses two separate CARs with different ligand binding targets; one CAR includes only the CD33 domain and the other CAR includes only the co-stimulatory domain(s). Dual CAR T cell activation requires co-expression of both targets on the tumor.

A safety CAR (sCAR) consists of an extracellular scFv fused to an intracellular inhibitory domain. SCAR T cells co-expressing a standard CAR become activated only when encountering target cells that possess the standard CAR target but lack the sCAR target.

The antigen recognition domain of the disclosed CAR is usually an scFv. There are however many alternatives. An antigen recognition domain from native T-cell receptor (TCR) alpha and beta single chains have been described, as have simple ectodomains (e.g. CD4 ectodomain to recognize HIV infected cells) and more exotic recognition components such as a linked cytokine (which leads to recognition of cells bearing the cytokine receptor). In fact almost anything that binds a given target with high affinity can be used as an antigen recognition region.

The endodomain is the business end of the CAR that after antigen recognition transmits a signal to the immune effector cell, activating at least one of the normal effector functions of the immune effector cell. Effector function of a T cell, for example, may be cytolytic activity or helper activity including the secretion of cytokines. Therefore, the endodomain may comprise the “intracellular signaling domain” of a T cell receptor (TCR) and optional co-receptors. While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal.

Cytoplasmic signaling sequences that regulate primary activation of the TCR complex that act in a stimulatory manner may contain signaling motifs which are known as immunoreceptor tyrosine-based activation motifs (ITAMs). Examples of ITAM containing cytoplasmic signaling sequences include those derived from CD8, CD3ζ, CD3δ, CD3γ, CD3ε, CD32 (Fc gamma Rlla), DAP10, DAP12, CD79a, CD79b, FcγRly, FcγRIIIy, FcεRIβ (FCERIB), and FcaRly (FCERIG).

In particular embodiments, the intracellular signaling domain is derived from CD3 zeta (CD3δ) (TCR zeta, GenBank accno. BAG36664.1). T-cell surface glycoprotein CD3 zeta (CD3δ) chain, also known as T-cell receptor T3 zeta chain or CD247 (Cluster of Differentiation 247), is a protein that in humans is encoded by the CD247 gene.

First-generation CARs typically had the intracellular domain from the CD3δ chain, which is the primary transmitter of signals from endogenous TCRs. Second-generation CARs add intracellular signaling domains from various costimulatory protein receptors (e.g., CD28, 41BB, ICOS) to the endodomain of the CAR to provide additional signals to the T cell. Preclinical studies have indicated that the second generation of CAR designs improves the antitumor activity of T cells. More recent, third-generation CARs combine multiple signaling domains to further augment potency. T cells grafted with these CARs have demonstrated improved expansion, activation, persistence, and tumor-eradicating efficiency independent of costimulatory receptor/ligand interaction (Imai C, et al. Leukemia 2004 18:676-84; Maher J, et al. Nat Biotechnol 2002 20:70-5).

For example, the endodomain of the CAR can be designed to comprise the CD3Z signaling domain by itself or combined with any other desired cytoplasmic domain(s) useful in the context of the CAR of the invention. For example, the cytoplasmic domain of the CAR can comprise a CD32 chain portion and a costimulatory signaling region. The costimulatory signaling region refers to a portion of the CAR comprising the intracellular domain of a costimulatory molecule. A costimulatory molecule is a cell surface molecule other than an antigen receptor or their ligands that is required for an efficient response of lymphocytes to an antigen. Examples of such molecules include CD27, CD28, 4-1BB (CD137), OX40, CD30, CD40, ICOS, lymphocyte function-associated antigen-1 (LFA-1), CD2, CD7, LIGHT, NKG2C, B7-H3, and a ligand that specifically binds with CD83, CD8, CD4, b2c, CD80, CD86, DAP10, DAP12, MyD88, BTNL3, and NKG2D. Thus, while the CAR is exemplified primarily with CD28 as the co-stimulatory signaling element, other costimulatory elements can be used alone or in combination with other co-stimulatory signaling elements.

In some embodiments, the CAR comprises a hinge sequence. A hinge sequence is a short sequence of amino acids that facilitates antibody flexibility (see, e.g., Woof et al., Nat. Rev. Immunol., 4 (2): 89-99 (2004)). The hinge sequence may be positioned between the antigen recognition moiety (e.g., anti-CD123 scFv) and the transmembrane domain. The hinge sequence can be any suitable sequence derived or obtained from any suitable molecule. In some embodiments, for example, the hinge sequence is derived from a CD8a molecule or a CD28 molecule.

The transmembrane domain may be derived either from a natural or from a synthetic source. Where the source is natural, the domain may be derived from any membrane-bound or transmembrane protein. For example, the transmembrane region may be derived from (i.e. comprise at least the transmembrane region(s) of) the alpha, beta or zeta chain of the T-cell receptor, CD28, CD3 epsilon, CD45, CD4, CD5, CD8 (e.g., CD8 alpha, CD8 beta), CD9, CD16, CD22, CD33, CD37, CD64, CD80, CD86, CD134, CD137, or CD154, KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), CD160, CD19, IL2R beta, IL2R gamma, IL7R a, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, and PAG/Cbp. Alternatively the transmembrane domain may be synthetic, in which case it will comprise predominantly hydrophobic residues such as leucine and valine. In some cases, a triplet of phenylalanine, tryptophan and valine will be found at each end of a synthetic transmembrane domain. A short oligo- or polypeptide linker, such as between 2 and 10 amino acids in length, may form the linkage between the transmembrane domain and the endoplasmic domain of the CAR.

In some embodiments, the CAR has more than one transmembrane domain, which can be a repeat of the same transmembrane domain, or can be different transmembrane domains.

In some embodiments, the CAR is a multi-chain CAR, as described in WO2015/039523, which is incorporated by reference for this teaching. A multi-chain CAR can comprise separate extracellular ligand binding and signaling domains in different transmembrane polypeptides. The signaling domains can be designed to assemble in juxtamembrane position, which forms flexible architecture closer to natural receptors, that confers optimal signal transduction. For example, the multi-chain CAR can comprise a part of an FCERI alpha chain and a part of an FCERI beta chain such that the FCERI chains spontaneously dimerize together to form a CAR.

In some embodiments, the recognition domain is a single chain variable fragment (scFv) antibody. The affinity/specificity of an scFv is driven in large part by specific sequences within complementarity determining regions (CDRs) in the heavy (VH) and light (VL) chain. Each VH and VL sequence will have three CDRs (CDR1, CDR2, CDR3).

In some embodiments, the recognition domain is derived from natural antibodies, such as monoclonal antibodies. In some cases, the antibody is human. In some cases, the antibody has undergone an alteration to render it less immunogenic when administered to humans. For example, the alteration comprises one or more techniques selected from the group consisting of chimerization, humanization, CDR-grafting, deimmunization, and mutation of framework amino acids to correspond to the closest human germline sequence.

Also disclosed are bi-specific CARs that target two different antigens. Also disclosed are CARs designed to work only in conjunction with another CAR that binds a different antigen, such as a tumor antigen. For example, in these embodiments, the endodomain of the disclosed CAR can contain only a signaling domain (SD) or a co-stimulatory signaling region (CSR), but not both. The second CAR (or endogenous T-cell) provides the missing signal if it is activated. For example, if the disclosed CAR contains an SD but not a CSR, then the immune effector cell containing this CAR is only activated if another CAR (or T-cell) containing a CSR binds its respective antigen. Likewise, if the disclosed CAR contains a CSR but not a SD, then the immune effector cell containing this CAR is only activated if another CAR (or T-cell) containing an SD binds its respective antigen.

Tumor antigens are proteins that are produced by tumor cells that elicit an immune response, particularly T-cell mediated immune responses. The additional antigen binding domain can be an antibody or a natural ligand of the tumor antigen. The selection of the additional antigen binding domain will depend on the particular type of cancer to be treated. Tumor antigens are well known in the art and include, for example, a glioma-associated antigen, carcinoembryonic antigen (CEA), EGFRvIII, IL-IIRa, IL-13Ra, EGFR, FAP, B7H3, Kit, CA LX, CS-1, MUC1, BCMA, bcr-abl, HER2, β-human chorionic gonadotropin, alphafetoprotein (AFP), ALK, CD19, TIM3, cyclin BI, lectin-reactive AFP, Fos-related antigen 1, ADRB3, thyroglobulin, EphA2, RAGE-1, RUI, RU2, SSX2, AKAP-4, LCK, OY-TESI, PAX5, SART3, CLL-1, fucosyl GM1, GloboH, MN-CA IX, EPCAM, EVT6-AML, TGS5, human telomerase reverse transcriptase, plysialic acid, PLAC1, RUI, RU2 (AS), intestinal carboxyl esterase, lewisY, sLe, LY6K, mut hsp70-2, M-CSF, MYCN, RhoC, TRP-2, CYPIBI, BORIS, prostase, prostate-specific antigen (PSA), PAX3, PAP, NY-ESO-1, LAGE-la, LMP2, NCAM, p53, p53 mutant, Ras mutant, gplOO, prostein, OR51E2, PANX3, PSMA, PSCA, Her2/neu, hTERT, HMWMAA, HAVCR1, VEGFR2, PDGFR-beta, survivin and telomerase, legumain, HPV E6, E7, sperm protein 17, SSEA-4, tyrosinase, TARP, WT1, prostate-carcinoma tumor antigen-1 (PCTA-1), ML-IAP, MAGE, MAGE-A1,MAD-CT-1, MAD-CT-2, MelanA/MART 1, XAGE1, ELF2M, ERG (TMPRSS2 ETS fusion gene), NA17, neutrophil elastase, sarcoma translocation breakpoints, NY-BR-1, ephnnB2, CD20, CD22, CD24, CD30, TIM3, CD38, CD44v6, CD97, CD171, CD179a, androgen receptor, FAP, insulin growth factor (IGF)-I, IGFII, IGF-I receptor, GD2, o-acetyl-GD2, GD3, GM3, GPRC5D, GPR20, CXORF61, folate receptor (FRa), folate receptor beta, ROR1, Flt3, TAG72, TN Ag, Tie 2, TEM1, TEM7R, CLDN6, TSHR, UPK2, and mesothelin. In a preferred embodiment, the tumor antigen is selected from the group consisting of folate receptor (FRa), mesothelin, EGFRvIII, IL-13Ra, CD123, CD19, TIM3, BCMA, GD2, CLL-1, CA-IX, MUCI, HER2, and any combination thereof.

Non-limiting examples of tumor antigens include the following: Differentiation antigens such as tyrosinase, TRP-1, TRP-2 and tumor-specific multilineage antigens such as MAGE-1, MAGE-3, BAGE, GAGE-1, GAGE-2, pi 5; overexpressed embryonic antigens such as CEA; overexpressed oncogenes and mutated tumor-suppressor genes such as p53, Ras, HER-2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7. Other large, protein-based antigens include TSP-180, MAGE-4, MAGE-5, MAGE-6, RAGE, NY-ESO, pl85erbB2, pl80erbB-3, c-met, nm-23H1, PSA, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCASI, SDCCAG1 6, TA-90\Mac-2 binding protein\cyclophilm C-associated protein, TAAL6, TAG72, TLP, TPS, GPC3, MUC16, LMP1, EBMA-1, BARF-1, CS1, CD319, HER1, B7H6, L1CAM, IL6, and MET.

### Nucleic Acids and Vectors

Also disclosed are polynucleotides and polynucleotide vectors encoding the disclosed chimeric receptors. Also disclosed are oligonucleotides for use in inserting the chimeric receptors into the genome of a T cell at a site that will disrupt Foxp1 expression or activity.

Nucleic acid sequences encoding the disclosed chimeric receptors, and regions thereof, can be obtained using recombinant methods known in the art, such as, for example by screening libraries from cells expressing the gene, by deriving the gene from a vector known to include the same, or by isolating directly from cells and tissues containing the same, using standard techniques. Alternatively, the gene of interest can be produced synthetically, rather than cloned.

### Immune Effector Cells

Also disclosed are immune effector cells that are engineered to express the disclosed chimeric receptors. These cells are preferably obtained from the subject to be treated (i.e. are autologous). However, in some embodiments, immune effector cell lines or donor effector cells (allogeneic) are used. Immune effector cells can be obtained from a number of sources, including peripheral blood mononuclear cells, bone marrow, lymph node tissue, cord blood, thymus tissue, tissue from a site of infection, ascites, pleural effusion, spleen tissue, and tumors. Immune effector cells can be obtained from blood collected from a subject using any number of techniques known to the skilled artisan, such as Ficoll™ separation. For example, cells from the circulating blood of an individual may be obtained by apheresis. In some embodiments, immune effector cells are isolated from peripheral blood lymphocytes by lysing the red blood cells and depleting the monocytes, for example, by centrifugation through a PERCOLL™ gradient or by counterflow centrifugal elutriation. A specific subpopulation of immune effector cells can be further isolated by positive or negative selection techniques. For example, immune effector cells can be isolated using a combination of antibodies directed to surface markers unique to the positively selected cells, e.g., by incubation with antibody-conjugated beads for a time period sufficient for positive selection of the desired immune effector cells. Alternatively, enrichment of immune effector cells population can be accomplished by negative selection using a combination of antibodies directed to surface markers unique to the negatively selected cells.

In some embodiments, the immune effector cells comprise any leukocyte involved in defending the body against infectious disease and foreign materials. For example, the immune effector cells can comprise lymphocytes, monocytes, macrophages, dentritic cells, mast cells, neutrophils, basophils, eosinophils, or any combinations thereof. For example, the immune effector cells can comprise T lymphocytes.

T cells or T lymphocytes can be distinguished from other lymphocytes, such as B cells and natural killer cells (NK cells), by the presence of a T-cell receptor (TCR) on the cell surface. They are called T cells because they mature in the thymus (although some also mature in the tonsils). There are several subsets of T cells, each with a distinct function.

T helper cells (TH cells) assist other white blood cells in immunologic processes, including maturation of B cells into plasma cells and memory B cells, and activation of cytotoxic T cells and macrophages. These cells are also known as CD4+ T cells because they express the CD4 glycoprotein on their surface. Helper T cells become activated when they are presented with peptide antigens by MHC class II molecules, which are expressed on the surface of antigen-presenting cells (APCs). Once activated, they divide rapidly and secrete small proteins called cytokines that regulate or assist in the active immune response. These cells can differentiate into one of several subtypes, including T_{H}1, T_{H}2, T_{H}3, T_{H}17, T_{H}9, or T_{FH}, which secrete different cytokines to facilitate a different type of immune response.

Cytotoxic T cells (T_{C} cells, or CTLs) destroy virally infected cells and tumor cells, and are also implicated in transplant rejection. These cells are also known as CD8⁺ T cells since they express the CD8 glycoprotein at their surface. These cells recognize their targets by binding to antigen associated with MHC class I molecules, which are present on the surface of all nucleated cells. Through IL-10, adenosine and other molecules secreted by regulatory T cells, the CD8+ cells can be inactivated to an anergic state, which prevents autoimmune diseases.

Memory T cells are a subset of antigen-specific T cells that persist long-term after an infection has resolved. They quickly expand to large numbers of effector T cells upon re-exposure to their cognate antigen, thus providing the immune system with “memory” against past infections. Memory cells may be either CD4⁺ or CD8⁺. Memory T cells typically express the cell surface protein CD45RO.

Regulatory T cells (T_{reg} cells), formerly known as suppressor T cells, are crucial for the maintenance of immunological tolerance. Their major role is to shut down T cell-mediated immunity toward the end of an immune reaction and to suppress auto-reactive T cells that escaped the process of negative selection in the thymus. Two major classes of CD4⁺ T_{reg} cells have been described—naturally occurring T_{reg} cells and adaptive T_{reg} cells.

Natural killer T (NKT) cells (not to be confused with natural killer (NK) cells) bridge the adaptive immune system with the innate immune system. Unlike conventional T cells that recognize peptide antigens presented by major histocompatibility complex (MHC) molecules, NKT cells recognize glycolipid antigen presented by a molecule called CD1d.

In some embodiments, the T cells comprise a mixture of CD4+ cells. In other embodiments, the T cells are enriched for one or more subsets based on cell surface expression. For example, in some cases, the T comprise are cytotoxic CD8⁺ T lymphocytes. In some embodiments, the T cells comprise γδ T cells, which possess a distinct T-cell receptor (TCR) having one γ chain and one δ chain instead of a and B chains.

Natural-killer (NK) cells are CD56⁺CD3⁻ large granular lymphocytes that can kill virally infected and transformed cells, and constitute a critical cellular subset of the innate immune system (Godfrey J, et al. Leuk Lymphoma 2012 53:1666-1676). Unlike cytotoxic CD8⁺ T lymphocytes, NK cells launch cytotoxicity against tumor cells without the requirement for prior sensitization, and can also eradicate MHC-I-negative cells (Narni-Mancinelli E, et al. Int Immunol 2011 23:427-431). NK cells are safer effector cells, as they may avoid the potentially lethal complications of cytokine storms (Morgan R A, et al. Mol Ther 2010 18:843-851), tumor lysis syndrome (Porter D L, et al. N Engl J Med 2011 365:725-733), and on-target, off-tumor effects. Although NK cells have a well-known role as killers of cancer cells, and NK cell impairment has been extensively documented as crucial for progression of MM (Godfrey J, et al. Leuk Lymphoma 2012 53:1666-1676; Fauriat C, et al. Leukemia 2006 20:732-733), the means by which one might enhance NK cell-mediated anti-MM activity has been largely unexplored prior to the disclosed CARs.

### Therapeutic Methods

Immune effector cells expressing the disclosed chimeric receptors can elicit an anti-tumor immune response against cancer cells. The anti-tumor immune response elicited by the disclosed chimeric cells may be an active or a passive immune response. In addition, the immune response may be part of an adoptive immunotherapy approach in which chimeric cells induce an immune response specific to the target antigen.

Adoptive transfer of immune effector cells expressing chimeric receptors is a promising anti-cancer therapeutic. Following the collection of a patient's immune effector cells, the cells may be genetically engineered to express the disclosed chimeric receptors while ablating Foxp1 according to the disclosed methods, then infused back into the patient.

The disclosed chimeric effector cells may be administered either alone, or as a pharmaceutical composition in combination with diluents and/or with other components such as IL-2, IL-15, or other cytokines or cell populations. Briefly, pharmaceutical compositions may comprise a target cell population as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions for use in the disclosed methods are in some embodiments formulated for intravenous administration. Pharmaceutical compositions may be administered in any manner appropriate treat tumors. The quantity and frequency of administration will be determined by such factors as the condition of the patient, and the severity of the patient's disease, although appropriate dosages may be determined by clinical trials.

When “an immunologically effective amount”, “an anti-tumor effective amount”, “an tumor-inhibiting effective amount”, or “therapeutic amount” is indicated, the precise amount of the compositions of the present invention to be administered can be determined by a physician with consideration of individual differences in age, weight, tumor size, extent of infection or metastasis, and condition of the patient (subject). It can generally be stated that a pharmaceutical composition comprising the T cells described herein may be administered at a dosage of 10⁴ to 10⁹ cells/kg body weight, such as 10⁵ to 10⁶ cells/kg body weight, including all integer values within those ranges. T cell compositions may also be administered multiple times at these dosages. The cells can be administered by using infusion techniques that are commonly known in immunotherapy (see, e.g., Rosenberg et al., New Eng. J. of Med. 319:1676, 1988). The optimal dosage and treatment regime for a particular patient can readily be determined by one skilled in the art of medicine by monitoring the patient for signs of disease and adjusting the treatment accordingly.

In certain embodiments, it may be desired to administer activated T cells to a subject and then subsequently re-draw blood (or have an apheresis performed), activate T cells therefrom according to the disclosed methods, and reinfuse the patient with these activated and expanded T cells. This process can be carried out multiple times every few weeks. In certain embodiments, T cells can be activated from blood draws of from 10 cc to 400 cc. In certain embodiments, T cells are activated from blood draws of 20 cc, 30 cc, 40 cc, 50 cc, 60 cc, 70 cc, 80 cc, 90 cc, or 100 cc. Using this multiple blood draw/multiple reinfusion protocol may serve to select out certain populations of T cells.

The administration of the disclosed compositions may be carried out in any convenient manner, including by injection, transfusion, or implantation. The compositions described herein may be administered to a patient subcutaneously, intradermally, intratumorally, intranodally, intramedullary, intramuscularly, by intravenous (i.v.) injection, or intraperitoneally. In some embodiments, the disclosed compositions are administered to a patient by intradermal or subcutaneous injection. In some embodiments, the disclosed compositions are administered by i.v. injection. The compositions may also be injected directly into a tumor, lymph node, or site of infection.

In certain embodiments, the disclosed chimeric cells are administered to a patient in conjunction with (e.g., before, simultaneously or following) any number of relevant treatment modalities, including but not limited to thalidomide, dexamethasone, bortezomib, and lenalidomide. In further embodiments, the chimeric cells may be used in combination with chemotherapy, radiation, immunosuppressive agents, such as cyclosporin, azathioprine, methotrexate, mycophenolate, and FK506, antibodies, or other immunoablative agents such as CAM PATH, anti-CD3 antibodies or other antibody therapies, cytoxin, fludaribine, cyclosporin, FK506, rapamycin, mycophenolic acid, steroids, FR901228, cytokines, and irradiation. In some embodiments, the CAR-modified immune effector cells are administered to a patient in conjunction with (e.g., before, simultaneously or following) bone marrow transplantation, T cell ablative therapy using either chemotherapy agents such as, fludarabine, external-beam radiation therapy (XRT), cyclophosphamide, or antibodies such as OKT3 or CAMPATH. In another embodiment, the cell compositions of the present invention are administered following B-cell ablative therapy such as agents that react with CD20, e.g., Rituxan. For example, in some embodiments, subjects may undergo standard treatment with high dose chemotherapy followed by peripheral blood stem cell transplantation. In certain embodiments, following the transplant, subjects receive an infusion of the expanded immune cells of the present invention. In an additional embodiment, expanded cells are administered before or following surgery.

The cancer of the disclosed methods can be any cell in a subject undergoing unregulated growth, invasion, or metastasis. In some aspects, the cancer can be any neoplasm or tumor for which radiotherapy is currently used. Alternatively, the cancer can be a neoplasm or tumor that is not sufficiently sensitive to radiotherapy using standard methods. Thus, the cancer can be a sarcoma, lymphoma, leukemia, carcinoma, blastoma, or germ cell tumor. A representative but non-limiting list of cancers that the disclosed compositions can be used to treat include lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, kidney cancer, lung cancers such as small cell lung cancer and non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, endometrial cancer, cervical cancer, cervical carcinoma, breast cancer, epithelial cancer, renal cancer, genitourinary cancer, pulmonary cancer, esophageal carcinoma, head and neck carcinoma, large bowel cancer, hematopoietic cancers; testicular cancer; colon and rectal cancers, prostatic cancer, and pancreatic cancer.

The disclosed chimeric cells can be used in combination with any compound, moiety or group which has a cytotoxic or cytostatic effect. Drug moieties include chemotherapeutic agents, which may function as microtubulin inhibitors, mitosis inhibitors, topoisomerase inhibitors, or DNA intercalators, and particularly those which are used for cancer therapy.

The disclosed chimeric cells can be used in combination with a checkpoint inhibitor. The two known inhibitory checkpoint pathways involve signaling through the cytotoxic T-lymphocyte antigen-4 (CTLA-4) and programmed-death 1 (PD-1) receptors. These proteins are members of the CD28-B7 family of cosignaling molecules that play important roles throughout all stages of T cell function. The PD-1 receptor (also known as CD279) is expressed on the surface of activated T cells. Its ligands, PD-L1 (B7-H1; CD274) and PD-L2 (B7-DC; CD273), are expressed on the surface of APCs such as dendritic cells or macrophages. PD-L1 is the predominant ligand, while PD-L2 has a much more restricted expression pattern. When the ligands bind to PD-1, an inhibitory signal is transmitted into the T cell, which reduces cytokine production and suppresses T-cell proliferation. Checkpoint inhibitors include, but are not limited to antibodies that block PD-1 (Nivolumab (BMS-936558 or MDX1106), CT-011, MK-3475), PD-L1 (MDX-1105 (BMS-936559), MPDL3280A, MSB0010718C), PD-L2 (rHlgM12B7), CTLA-4 (Ipilimumab (MDX-010)), Tremelimumab (CP-675,206), IDO, B7-H3 (MGA271), B7-H4, TIM3, LAG-3 (BMS-986016).

Human monoclonal antibodies to programmed death 1 (PD-1) and methods for treating cancer using anti-PD-1 antibodies alone or in combination with other immunotherapeutics are described in U.S. Pat. No. 8,008,449, which is incorporated by reference for these antibodies. Anti-PD-L1 antibodies and uses therefor are described in U.S. Pat. No. 8,552,154, which is incorporated by reference for these antibodies. Anticancer agent comprising anti-PD-1 antibody or anti-PD-L1 antibody are described in U.S. Pat. No. 8,617,546, which is incorporated by reference for these antibodies.

In some embodiments, the PDL1 inhibitor comprises an antibody that specifically binds PDL1, such as BMS-936559 (Bristol-Myers Squibb) or MPDL3280A (Roche). In some embodiments, the PD1 inhibitor comprises an antibody that specifically binds PD1, such as lambrolizumab (Merck), nivolumab (Bristol-Myers Squibb), or MEDI4736 (AstraZeneca). Human monoclonal antibodies to PD-1 and methods for treating cancer using anti-PD-1 antibodies alone or in combination with other immunotherapeutics are described in U.S. Pat. No. 8,008,449, which is incorporated by reference for these antibodies. Anti-PD-L1 antibodies and uses therefor are described in U.S. Patent No. 8,552, 154, which is incorporated by reference for these antibodies. Anticancer agent comprising anti-PD-1 antibody or anti-PD-L1 antibody are described in U.S. Pat. No. 8,617,546, which is incorporated by reference for these antibodies.

The disclosed chimeric cells can be used in combination with other cancer immunotherapies. There are two distinct types of immunotherapy: passive immunotherapy uses components of the immune system to direct targeted cytotoxic activity against cancer cells, without necessarily initiating an immune response in the patient, while active immunotherapy actively triggers an endogenous immune response. Passive strategies include the use of the monoclonal antibodies (mAbs) produced by B cells in response to a specific antigen. The development of hybridoma technology in the 1970s and the identification of tumor-specific antigens permitted the pharmaceutical development of mAbs that could specifically target tumor cells for destruction by the immune system. Thus far, mAbs have been the biggest success story for immunotherapy; the top three best-selling anticancer drugs in 2012 were mAbs. Among them is rituximab (Rituxan, Genentech), which binds to the CD20 protein that is highly expressed on the surface of B cell malignancies such as non-Hodgkin's lymphoma (NHL). Rituximab is approved by the FDA for the treatment of NHL and chronic lymphocytic leukemia (CLL) in combination with chemotherapy. Another important mAb is trastuzumab (Herceptin; Genentech), which revolutionized the treatment of HER2 (human epidermal growth factor receptor 2)-positive breast cancer by targeting the expression of HER2.

Generating optimal “killer” CD8 T cell responses also requires T cell receptor activation plus co-stimulation, which can be provided through ligation of tumor necrosis factor receptor family members, including OX40 (CD134) and 4-1BB (CD137). OX40 is of particular interest as treatment with an activating (agonist) anti-OX40 mAb augments T cell differentiation and cytolytic function leading to enhanced anti-tumor immunity against a variety of tumors.

In some embodiments, such an additional therapeutic agent may be selected from an antimetabolite, such as methotrexate, 6-mercaptopurine, 6-thioguanine, cytarabine, fludarabine, 5-fluorouracil, decarbazine, hydroxyurea, asparaginase, gemcitabine or cladribine.

In some embodiments, such an additional therapeutic agent may be selected from an alkylating agent, such as mechlorethamine, thioepa, chlorambucil, melphalan, carmustine (BSNU), lomustine (CCNU), cyclophosphamide, busulfan, dibromomannitol, streptozotocin, dacarbazine (DTIC), procarbazine, mitomycin C, cisplatin and other platinum derivatives, such as carboplatin.

In some embodiments, such an additional therapeutic agent is a targeted agent, such as ibrutinib or idelalisib.

In some embodiments, such an additional therapeutic agent is an epigenetic modifier such as azacitdine or vidaza.

In some embodiments, such an additional therapeutic agent may be selected from an anti-mitotic agent, such as taxanes, for instance docetaxel, and paclitaxel, and vinca alkaloids, for instance vindesine, vincristine, vinblastine, and vinorelbine.

In some embodiments, such an additional therapeutic agent may be selected from a topoisomerase inhibitor, such as topotecan or irinotecan, or a cytostatic drug, such as etoposide and teniposide.

In some embodiments, such an additional therapeutic agent may be selected from a growth factor inhibitor, such as an inhibitor of ErbBI (EGFR) (such as an EGFR antibody, e.g. zalutumumab, cetuximab, panitumumab or nimotuzumab or other EGFR inhibitors, such as gefitinib or erlotinib), another inhibitor of ErbB2 (HER2/neu) (such as a HER2 antibody, e.g. trastuzumab, trastuzumab-DM I or pertuzumab) or an inhibitor of both EGFR and HER2, such as lapatinib).

In some embodiments, such an additional therapeutic agent may be selected from a tyrosine kinase inhibitor, such as imatinib (Glivec, Gleevec STI571) or lapatinib.

Therefore, in some embodiments, a disclosed antibody is used in combination with ofatumumab, zanolimumab, daratumumab, ranibizumab, nimotuzumab, panitumumab, hu806, daclizumab (Zenapax), basiliximab (Simulect), infliximab (Remicade), adalimumab (Humira), natalizumab (Tysabri), omalizumab (Xolair), efalizumab (Raptiva), and/or rituximab.

In some embodiments, a therapeutic agent for use in combination with chimeric cells for treating the disorders as described above may be an anti-cancer cytokine, chemokine, or combination thereof. Examples of suitable cytokines and growth factors include IFNy, IL-2, IL-4, IL-6, IL-7, IL-10, IL-12, IL-13, IL-15, IL-18, IL-23, IL-24, IL-27, IL-28a, IL-28b, IL-29, KGF, IFNa (e.g., INFa2b), IFN, GM-CSF, CD40L, Flt3 ligand, stem cell factor, ancestim, and TNFa. Suitable chemokines may include Glu-Leu-Arg (ELR)-negative chemokines such as IP-10, MCP-3, MIG, and SDF-la from the human CXC and C-C chemokine families. Suitable cytokines include cytokine derivatives, cytokine variants, cytokine fragments, and cytokine fusion proteins.

In some embodiments, a therapeutic agent for use in combination with chimeric cells for treating the disorders as described above may be a cell cycle control/apoptosis regulator (or “regulating agent”). A cell cycle control/apoptosis regulator may include molecules that target and modulate cell cycle control/apoptosis regulators such as (i) cdc-25 (such as NSC 663284), (ii) cyclin-dependent kinases that overstimulate the cell cycle (such as flavopiridol (L868275, HMR1275), 7-hydroxystaurosporine (UCN-01, KW-2401), and roscovitine (R-roscovitine, CYC202)), and (iii) telomerase modulators (such as BIBR1532, SOT-095, GRN163 and compositions described in for instance U.S. Pat. Nos. 6,440,735 and 6,713,055). Non-limiting examples of molecules that interfere with apoptotic pathways include TNF-related apoptosis-inducing ligand (TRAIL)/apoptosis-2 ligand (Apo-2L), antibodies that activate TRAIL receptors, IFNs, and anti-sense Bcl-2.

In some embodiments, a therapeutic agent for use in combination with chimeric cells for treating the disorders as described above may be a hormonal regulating agent, such as agents useful for anti-androgen and anti-estrogen therapy. Examples of such hormonal regulating agents are tamoxifen, idoxifene, fulvestrant, droloxifene, toremifene, raloxifene, diethylstilbestrol, ethinyl estradiol/estinyl, an antiandrogene (such as flutaminde/eulexin), a progestin (such as such as hydroxyprogesterone caproate, medroxy-progesterone/provera, megestrol acepate/megace), an adrenocorticosteroid (such as hydrocortisone, prednisone), luteinizing hormone-releasing hormone (and analogs thereof and other LHRH agonists such as buserelin and goserelin), an aromatase inhibitor (such as anastrazole/arimidex, aminoglutethimide/cytraden, exemestane) or a hormone inhibitor (such as octreotide/sandostatin).

In some embodiments, a therapeutic agent for use in combination with chimeric cells for treating the disorders as described above may be an anti-cancer nucleic acid or an anti-cancer inhibitory RNA molecule.

Combined administration, as described above, may be simultaneous, separate, or sequential. For simultaneous administration the agents may be administered as one composition or as separate compositions, as appropriate.

In some embodiments, the disclosed chimeric cells are administered in combination with radiotherapy. Radiotherapy may comprise radiation or associated administration of radiopharmaceuticals to a patient is provided. The source of radiation may be either external or internal to the patient being treated (radiation treatment may, for example, be in the form of external beam radiation therapy (EBRT) or brachytherapy (BT)). Radioactive elements that may be used in practicing such methods include, e.g., radium, cesium-137, iridium-192, americium-241, gold-198, cobalt-57, copper-67, technetium-99, iodide-123, iodide-131, and indium-111.

### EXAMPLE EMBODIMENTS

Embodiment 1. A chimeric cell expressing a chimeric receptor,
wherein the chimeric receptor is encoded by a transgene, and
wherein the transgene is inserted in the genome of the cell at a location that disrupts expression or activity of an endogenous X-box binding protein 1 (XBP1) and/or protein kinase R (PKR)-like endoplasmic reticulum kinase (PERK).

Embodiment 2. The chimeric cell of embodiment 1, wherein the chimeric receptor is a chimeric antigen receptor (CAR) polypeptide.

Embodiment 3. The chimeric cell of embodiment 2, wherein the chimeric receptor comprises two subunits of a follicule-stimulating hormone (FSH).

Embodiment 4. The chimeric cell of embodiment 3, wherein the chimeric receptor comprises the amino acid sequence SEQ ID NO:1, or a variant thereof having at least 90% sequence identity to SEQ ID NO:1

Embodiment 5. The chimeric cell of any one of embodiments 1 to 4, wherein the cell is selected from the group consisting of an αβT cell, γδT cell, a Natural Killer (NK) cells, a Natural Killer T (NKT) cell, a B cell, an innate lymphoid cell (ILC), a cytokine induced killer (CIK) cell, a cytotoxic T lymphocyte (CTL), a lymphokine activated killer (LAK) cell, a regulatory T cell, or any combination thereof.

Embodiment 6. The chimeric cell of any one of embodiments 3 to 5, wherein the cell exhibits an anti-tumor immunity when the antigen binding domain of the chimeric receptor binds to an FSH-receptor positive ovarian tumor.

Embodiment 7. A method of providing an anti-cancer immunity in a subject, comprising administering to the subject an effective amount of the chimeric cell of any one of embodiments 1 to 6, thereby providing an anti-tumor immunity in the subject.

Embodiment 8. The method of embodiment 7, wherein the chimeric receptor comprises two subunits of a follicule-stimulating hormone (FSH), and wherein the subject has an FSH-receptor positive ovarian tumor.

Embodiment 9. The method of embodiment 7 or 8, further comprising administering to the subject a checkpoint inhibitor.

Embodiment 10. The method of embodiment 9, wherein the checkpoint inhibitor comprises an anti-PD-1 antibody, anti-PD-L1 antibody, anti-CTLA-4 antibody, or a combination thereof.

A number of embodiments of the invention have been described. Nevertheless, it will be understood that various modifications may be made without departing from the spirit and scope of the invention. Accordingly, other embodiments are within the scope of the following claims.

### Examples

### Example 1: XBP1 Ablation

Generation of RNP Using Synthetic crRNA and tracrRNA

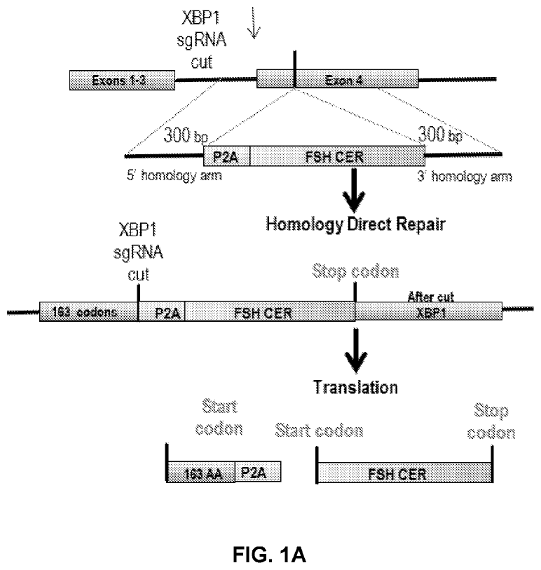

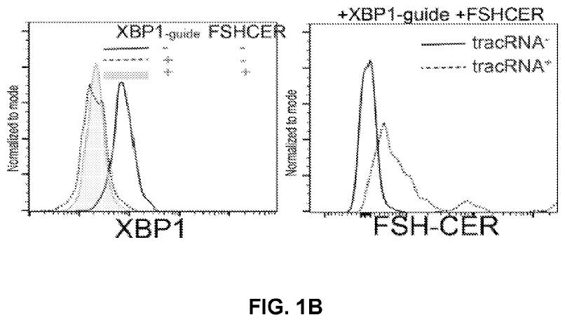

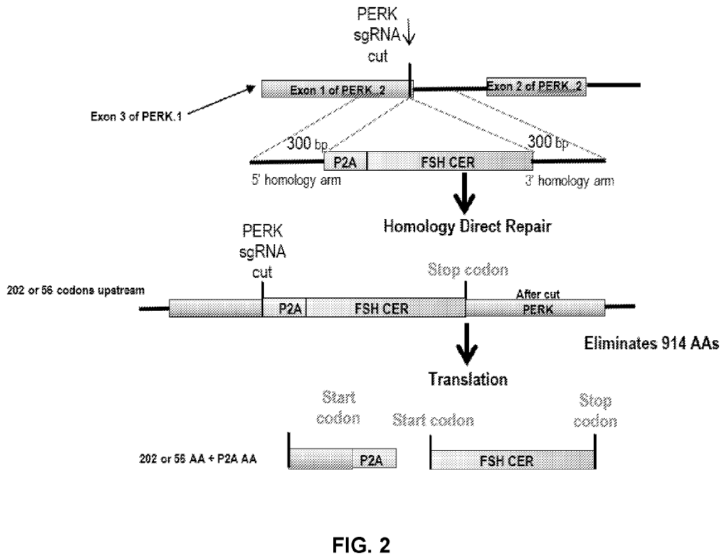

Non-viral integration of FSH-CER constructs with concurrent ablation of ER stress. Emerging studies indicated that CAR/TCR/CER constructs can be effectively expressed in human T cells while avoiding viral vectors by using CRISPR and homology-directed repair (HDR) (Roth, T. L., et al. Nature 559, 405-409 (2018)). To circumvent the use of viral vectors and express our FSH-CER constructs while targeting ER stress, a system was designed to integrate a 1,386 bp DNA cassette immediately at exon 4 of XBP1. This cassette encodes the subunits of the human FSH-CER sequence, downstream of a self-excising 2A peptide. The cellular machinery that drives the constitutive expression of XBP1 in T cells ensures the expression of the FSH-CER, while the stop codon at position 1,383 and relatively long sequence of the construct will prevent the transcription/translation of both spliced and non-spliced forms of XBP1 (FIG. 1A). Importantly, XBP1-deficient T cells do not show defects in proliferation, while exhibiting superior anti-tumor activity (Song, M., et al. Nature 562, 423-428 (2018)). A fluorescently-labeled (550 nm) tracRNA, plus a published crRNA oligo (Chopra, S., et al. Science 365 (2019)) with the XBP1-specific guide was used followed by the tracrRNA fusion domain. Co-electroporation of CAS9-based ctRNP complexes with an HDR template encoding P2A+FSH-CER+two 300 bp homology arms, designed to integrate FSH-CER in frame with XBP1 gene at exon 4, was performed using our Neon protocol (Roth, T. L., et al. Nature 559, 405-409 (2018); Osborn, M. J., et al. Mol Ther 24, 570-581 (2016); Rahdar, M., et al. Proc Natl Acad Sci USA 112, E7110-7117 (2015)). As proof-of-concept, this approach reduced XBP1s in T cells, while turning-on the CGa subunit of FSH on the T cell surface (FIG. 1B). Thus, Xbp1; FSH-CER⁻/Xbp1⁺ FSH-CER-T cells will be expanded in response to: 1) our FSHR⁺ artificial APCs (aAPCs) (Cubillos-Ruiz, J. R., et al. COncotarget 1, 329-328 (2010); Huarte, E., et al. Blood 112, 1259-1268 (2008)); 2) aAPCs coated with anti-CD3/CD28 mAbs; or 3) anti-CD3/CD28 beads. In parallel, anti-CD3/CD28-primed T cells are transduced mimicking clinical protocols. For all groups, acquisition of memory phenotypes (i.e., CD27, CCR7, CD122); stem-like attributes (i.e., TCF1, CXCR5); superior metabolic profile (i.e., OxPhos, glycolysis, fatty acids); higher CD8/CD4 ratios; elevated expansion/survival; and increased in vivo effectiveness will be compared as a function of: 1) XBP1 ablation; and 2) T cell priming through 4-1BB vs. CD28 (FSH-CER).

Order (fluorescently labeled; 550 nm) tracRNA from IDT DNA (Alt-R® CRISPR-Cas9 tracrRNA, ATTO™ 550; e.g., Catalog #1075928).

Order the crRNA (RNA) oligo from IDT DNA or Sigma, with the following sequence, with the guide specific for XBP1, plus the the 16 nt tracrRNA fusion domain: (or order from pre-designed sequences from IDT DNA:

(SEQ ID NO: 3)
UGCACGUAGUCUGAGUG↓CUGGUUUUAGAGCUAGAAA.

Form guide RNA complexes by combining the crRNA and tracrRNA in equal molar amounts in IDT Duplex Buffer (30 mM HEPES, pH 7.5, 100 mM Potassium Acetate) at 1 μM concentration by heating the oligos to 95 °C and slowly cooling to room temperature. Keep working stocks of crRNAs and tracrRNA at 10 UM concentration in TE (10 mM Tris, pH 7.5, 0.1 mM EDTA), in which case a mix 1 μL of crRNA and 1 μL of tracrRNA with 8 μL of Duplex Buffer is made.

While not always necessary, the heat/cool step improves performance for approximately 10% of target sites.

Excess of the 1 UM crRNA: tracrRNA complex can be stored for later use at 4 °C, −20 °C or −80 °C for at least 3 months.

Dilute Alt-R™ 3NLS Cas9 Nuclease (Integrated DNA Technologies) from stock 61 μM (10 mg/mL) to 1 μM in Opti-MEM (Thermo Fisher Scientific, Carlsbad, CA USA). Final transfections will employ 10 nM ctRNP complex.

The ctRNP complex is prepared by combining 5.25 μL of the 1 μM crRNA: tracrRNA complex with 5.25 L of the 1 M diluted stock of Cas9 protein. (Note: excess of the 1 μM RNP complex can be made and stored for later use at 4 °C or −80 °C for at least 3 months). Add 77 μL of Opti-MEM medium, bringing the final volume to 87.5 μL, yielding a final 60 nM concentration of RNP complex.

Incubate this mixture at room temperature for 5 min.

### Components for Electroporation

ECM830 Electro Square Wave Porator (Harvard Apparatus BTX, MA, USA)
2 mm cuvette (Catalog no. 1652086, Biorad, Hercules, CA, USA)
RNA introduction into target cells can be carried out using other available electroporation instruments that are commercially available, including, but not limited to Amaxa Nucleofactor-II (Amaxa Biosystems, Cologne, Germany), Gene Pulser Xcell (Biorad, Denver, CO, USA) or Multiporator (Eppendorf, Hamburg, Germany).

The protocol can use a Neon Transfection system (Thermo), buffer T 1400V, 10 ms and 3 pulses of electroporation.

Generation of ssDNA HDRT (Try First with dsDNA HDRT)

Double-stranded DNA HDRT production: Order through GenScript the homology arms and the desired hFSH_CER insert. The plasmid is used as template for high-output PCR amplification. PCR amplicons (the dsDNA HDRT) is purified and eluted into a final volume of 3 μl H₂O per 100 μl of PCR reaction input. Concentrations of HDRTs are determined by nanodrop using a 1:20 dilution.

Single-stranded DNA HDRT production by exonuclease digestion: To produce long ssDNA as HDR templates, the DNA of interest was amplified via PCR using one regular, non-modified PCR primer and a second phosphorylated PCR primer. The DNA strand that will be amplified using the phosphorylated primer will be the strand that will be degraded using this method. This makes it possible to prepare either a single-stranded sense or single-stranded antisense DNA using the respective phosphorylated PCR primer. To produce the ssDNA strand of interest, the phosphorylated strand of the PCR product was degraded by treatment with two enzymes, Strandase Mix A and Strandase Mix B, for 5 min (per 1 kb) at 37 °C, respectively. Enzymes were deactivated by a 5 min incubation at 80 °C. The resulting ssDNA HDR templates are purified and eluted in H₂O. A more detailed protocol for the Guide-it Long ssDNA Production System (Takara Bio, 632644) can be found at the manufacturer's website.

### Electroporation Method

Wash T cells to be electroporated (X 3 times) using Opti-MEM I: Reduced serum medium (Catalog no. 31985, Gibco, Grand Island, NY, USA). Centrifuge cells at 300xg for 5 minutes at 4 °C.

Carefully discard supernatant and resuspend cell pellet in fresh Opti-MEM media at 1×10⁸ cells/ml. For each electroporation, aliquot 1×10⁷ cells in a 100 μl of Opti-MEM. Keep cells on ice until use.

Pre-configure the electroporator by setting the voltage to 500V and time to 1000μ-seconds.

Prewarm R10 media to 37 °C and add 10 ml of the media to a T25 flask.

Place the cells in 100 μL into a 2 mm cuvette.

Add 10 μL of RNP complex plus 2 μL HDRT with the 100 μl aliquot of cells. Uniformly mix by gentle pipetting.

Place the cuvette into the electroporator cassette, tighten the electrodes around the metal plates of the cuvette and initiate the electric pulse.

Immediately transfer the contents of the cuvette into the T25 flask containing R10.

Rinse the cuvette once with fresh R10 to maximize recovery of electroporated cells.

Place the cells in a 37 °C. CO2 incubator until further use.

After 25 hrs, positively electroporated cells can be sorted by using the fluorescent label on the tracRNA (550 nm), after 24 hrs. To verify expression of the CER on the cell surface FACS analysis using anti-FSH Abs can be used, or the expression of FSH can be tested by Q-PCR using the following primers:

TABLE X
Primers for 137 bp

| OLIGO | start | len | tm | gc % | any_th | 3′_th | hairpin | seq |
|---|---|---|---|---|---|---|---|---|
| LEFT | 73 | 23 | 61.73 | 47.83 | 0.00 | 0.00 | 0.00 | CTGACCAACATCACAATCGCCAT |
| PRIMER | | | | | | | | (SEQ ID NO: 4) |
| RIGHT | 209 | 23 | 61.68 | 52.17 | 0.00 | 0.00 | 0.00 | GTTTTCTGGATCTTGGGTCTGGC |
| PRIMER | | | | | | | | (SEQ ID NO: 5) |

TABLE X
Primers for 140 bp

| Sequence (5′->3′) | Template strand | Tm |
|---|---|---|
| Forward primer | ACAGAGTGACCGTGATGGGC | 54 |
| | (SEQ ID NO: 6) | |
| Reverse primer | CTGGGAGGCGATTGTAGGGG | 155 |
| | (SEQ ID NO: 7) | |

Electropotared, CRISPRed T cells are then expanded against irradiated K562 cells transduced with human FSHR (1:10 ratio), in R10 media with II-2 (300 UI/ml).

### HDRT Sequence (XBP1+P2A+Tg; Insert is 1440 bp)

5′-arm:

(SEQ ID NO: 8)
CACGCACCTGAGCCCCGAGGAGAAGGCGCTGAGGAGGAAACTGAAAAAC
AGAGTAGCAGCTCAGACTGCCAGAGATCGAAAGAAGGCTCGAATGAGTG
AGCTGGAACAGCAAGTGGTAGATTTAGAAGAAGAGAACCAAAAACTTTT
GCTAGAAAATCAGCTTTTACGAGAGAAAACTCATGGCCTTGTAGTTGAG
AACCAGGAGTTAAGACAGCGCTTGGGGATGGATGCCCTGGTTGCTGAAG
AGGAGGCGGAAGCCAAGGGGAATGAAGTGAGGCCAGTGGCCGGGTCTGC
TGAGTC.

g+P2A (Inserted at Place of CRISPR Excision (GTCITGG):

(SEQ ID NO: 9)
TGGCCACGAACTTCTCCCTGTTAAAGCAAGCAGGAGACGTGGAAGAAAAC
CCCGGTCCC.

### FSHCER:

(SEQ ID NO: 10)
ATGAAGACCCTGCAGTTCTTCTTCCTGTTCTGCTGCTGGAAGGCCATCTG
CTGCAACAGCTGCGAGCTGACCAACATCACAATCGCCATCGAGAAAGAGG
AATGCCGGTTCTGCATCAGCATCAACACCACTTGGTGCGCCGGCTACTGC
TACACCCGGGACCTGGTGTACAAGGACCCCGCCAGACCCAAGATCCAGAA
AACCTGCACCTTCAAAGAACTGGTGTACGAGACAGTGCGGGTGCCCGGAT
GTGCCCACCATGCCGATAGCCTGTACACCTACCCTGTGGCCACCCAGTGT
CACTGCGGCAAGTGCGATAGCGACAGCACCGATTGCACCGTGCGGGGACT
GGGCCCTAGCTACTGTAGCTTCGGCGAGATGAAGGAAGGCGGCGGATCTG
GCGGAGGAAGCGGAGGGGGATCTGGGGGGGGAGCACCTGATGTGCAGGAT
TGCCCTGAGTGCACCCTGCAGGAAAACCCATTCTTCAGCCAGCCTGGCGC
CCCTATCCTGCAGTGCATGGGCTGCTGCTTCAGCAGAGCCTACCCCACCC
CCCTGCGGAGCAAGAAAACCATGCTGGTGCAGAAAAACGTGACCAGCGAG
AGCACCTGTTGCGTGGCCAAGAGCTACAACAGAGTGACCGTGATGGGCGG
CTTCAAGGTGGAAAACCACACCGCCTGCCACTGCAGCACATGCTACTACC
ACAAGAGCGCTAGCACCACCACCCCTGCCCCTAGACCTCCAACACCCGCC
CCTACAATCGCCTCCCAGCCTCTGTCTCTGAGGCCCGAGGCTTGTAGACC
AGCTGCTGGCGGAGCCGTGCACACCAGAGGACTGGATTTCGCCTGCGACA
TCTACATCTGGGCCCCTCTGGCCGGCACATGTGGCGTGCTGCTGCTGAGC
CTCGTGATCACCCTGTACTGCAAGCGGGGCAGAAAGAAGCTGCTGTACAT
CTTCAAGCAGCCCTTCATGCGGCCCGTGCAGACCACCCAGGAAGAGGACG
GCTGCTCCTGCAGATTCCCCGAAGAGGAAGAGGGGGGCTGCGAACTGAGA
GTGAAGTTCAGCAGAAGCGCCGACGCCCCTGCCTACAAGCAGGGCCAGAA
CCAGCTGTACAACGAGCTGAACCTGGGCAGACGGGAAGAGTACGACGTGC
TGGACAAGCGGAGAGGCAGGGACCCTGAGATGGGCGGAAAGCCCAGACGG
AAGAACCCCCAGGAAGGCCTGTATAACGAACTGCAGAAAGACAAGATGGC
CGAGGCCTACAGCGAGATCGGAATGAAGGGCGAGCGGAGAAGAGGCAAGG
GCCACGATGGCCTGTACCAGGGCCTGAGCACCGCCACCAAGGACACCTAT
GACGCCCTGCACATGCAGGCCCTGCCCCCCAGATAA.

3′-arm:

(SEQ ID NO: 11)
CGCAGCACTCAGACTACGTGCACCTCTGCAGCAGGTGCAGGCCCAGTTGT
CACCCCTCCAGAACATCTCCCCATGGATTCTGGCGGTATTGACTCTTCAG
ATTCAGAGTCTGATATCCTGTTGGGCATTCTGGACAACTTGGACCCAGTC
ATGTTCTTCAAATGCCCTTCCCCAGAGCCTGCCAGCCTGGAGGAGCTCCC
AGAGGTCTACCCAGAAGGACCCAGTTCCTTACCAGCCTCCCTTTCTCTGT
CAGTGGGGACGTCATCAGCCAAGCTGGAAGCCATTAATGAACTAATTCGT
T.

HDRT Sequence to be Ordered from Genescript:

(SEQ ID NO: 12)
cacgcacctgagccccgaggagaaggcgctgaggaggaaactgaaaaaca
gagtagcagctcagactgccagagatcgaaagaaggctcgaatgagtgag
ctggaacagcaagtggtagatttagaagaagagaaccaaaaacttttgct
agaaaatcagcttttacgagagaaaactcatggccttgtagttgagaacc
aggagttaagacagcgcttggggatggatgccctggttgctgaagaggag
gcggaagccaaggggaatgaagtgaggccagtggccgggtctgctgagtc
tggccacgaacttctccctgttaaagCaagcaggagacgtggaagaaaac
cccggtcccATGAAGACCCTGCAGTTCTTCTTCCTGTTCTGCTGCTGGAA
GGCCATCTGCTGCAACAGCTGCGAGCTGACCAACATCACAATCGCCATCG
AGAAAGAGGAATGCCGGTTCTGCATCAGCATCAACACCACTTGGTGCGCC
GGCTACTGCTACACCCGGGACCTGGTGTACAAGGACCCCGCCAGACCCAA
GATCCAGAAAACCTGCACCTTCAAAGAACTGGTGTACGAGACAGTGCGGG
TGCCCGGATGTGCCCACCATGCCGATAGCCTGTACACCTACCCTGTGGCC
ACCCAGTGTCACTGCGGCAAGTGCGATAGCGACAGCACCGATTGCACCGT
GCGGGGACTGGGCCCTAGCTACTGTAGCTTCGGCGAGATGAAGGAAGGCG
GCGGATCTGGCGGAGGAAGCGGAGGGGGATCTGGGGGCGGAGCACCTGAT
GTGCAGGATTGCCCTGAGTGCACCCTGCAGGAAAACCCATTCTTCAGCCA
GCCTGGCGCCCCTATCCTGCAGTGCATGGGCTGCTGCTTCAGCAGAGCCT
ACCCCACCCCCCTGCGGAGCAAGAAAACCATGCTGGTGCAGAAAAACGTG
ACCAGCGAGAGCACCTGTTGCGTGGCCAAGAGCTACAACAGAGTGACCGT
GATGGGCGGCTTCAAGGTGGAAAACCACACCGCCTGCCACTGCAGCACAT
GCTACTACCACAAGAGCGCTAGCACCACCACCCCTGCCCCTAGACCTCCA
ACACCCGCCCCTACAATCGCCTCCCAGCCTCTGTCTCTGAGGCCCGAGGC
TTGTAGACCAGCTGCTGGCGGAGCCGTGCACACCAGAGGACTGGATTTCG
CCTGCGACATCTACATCTGGGCCCCTCTGGCCGGCACATGTGGCGTGCTG
CTGCTGAGCCTCGTGATCACCCTGTACTGCAAGCGGGGCAGAAAGAAGCT
GCTGTACATCTTCAAGCAGCCCTTCATGCGGCCCGTGCAGACCACCCAGG
AAGAGGACGGCTGCTCCTGCAGATTCCCCGAAGAGGAAGAGGGGGGCTGC
GAACTGAGAGTGAAGTTCAGCAGAAGCGCCGACGCCCCTGCCTACAAGCA
GGGCCAGAACCAGCTGTACAACGAGCTGAACCTGGGCAGACGGGAAGAGT
ACGACGTGCTGGACAAGCGGAGAGGCAGGGACCCTGAGATGGGCGGAAAG
CCCAGACGGAAGAACCCCCAGGAAGGCCTGTATAACGAACTGCAGAAAGA
CAAGATGGCCGAGGCCTACAGCGAGATCGGAATGAAGGGCGAGCGGAGAA
GAGGCAAGGGCCACGATGGCCTGTACCAGGGCCTGAGCACCGCCACCAAG
GACACCTATGACGCCCTGCACATGCAGGCCCTGCCCCCCAGATAAcgcag
cactcagactacgtgcacctctgcagcaggtgcaggcccagttgtcaccc
ctccagaacatctccccatggattctggcggtattgactcttcagattca
gagtctgatatcctgttgggcattctggacaacttggacccagtcatgtt
cttcaaatgcccttccccagagcctgccagcctggaggagctcccagagg
tctacccagaaggacccagttccttaccagcctccctttctctgtcagtg
gggacgtcatcagccaagctggaagccattaatgaactaattcgtt.

### Example 2: PERK Ablation

Generation of RNP Using Synthetic crRNA and tracrRNA

Order (fluorescently labeled; 550 nm) tracRNA from IDT DNA (Alt-R® CRISPR-Cas9 tracrRNA, ATTO™ 550; e.g., Catalog #1075928).

Order the crRNA (RNA) oligo from IDT DNA or Sigma, with the following sequence, with the guide specific for PERK, plus the the 16 nt tracrRNA fusion domain: (or order from pre-designed sequences from IDT DNA:

(SEQ ID NO: 18)
UAUGGACUCAGUGCAUAUAGGUUUUAGAGCUAGAAA.

Form guide RNA complexes by combining the crRNA and tracrRNA in equal molar amounts in IDT Duplex Buffer (30 mM HEPES, pH 7.5, 100 mM Potassium Acetate) at 1 μM concentration by heating the oligos to 95 °C and slowly cooling to room temperature. Keep working stocks of crRNAs and tracrRNA at 10 μM concentration in TE (10 mM Tris, pH 7.5, 0.1 mM EDTA), in which case a mix 1 μL of crRNA and 1 μL of tracrRNA with 8 μL of Duplex Buffer is made. While not always necessary, the heat/cool step improves performance for approximately 10% of target sites.

Excess of the 1 UM crRNA: tracrRNA complex can be stored for later use at 4 °C, −20 °C or −80 °C for at least 3 months.

Dilute Alt-R™ 3NLS Cas9 Nuclease (Integrated DNA Technologies) from stock 61 μM (10 mg/mL) to 1 μM in Opti-MEM (Thermo Fisher Scientific, Carlsbad, CA USA). Final transfections will employ 10 nM ctRNP complex.

The ctRNP complex is prepared by combining 5.25 μL of the 1 μM crRNA: tracrRNA complex with 5.25 μL of the 1 μM diluted stock of Cas9 protein. (Note: excess of the 1 μM RNP complex can be made and stored for later use at 4 °C or −80 °C for at least 3 months.).Add 77 μL of Opti-MEM medium, bringing the final volume to 87.5 μL, yielding a final 60 nM concentration of RNP complex.

Incubate this mixture at room temperature for 5 min.

Generation of ssDNA HDRT (Try First with dsDNA HDRT)

Double-stranded DNA HDRT production: Order through GenScript the homology arms and the desired hFSH_CER insert. The plasmid is used as template for high-output PCR amplification. PCR amplicons (the dsDNA HDRT) is purified and eluted into a final volume of 3 μl H2O per 100 μl of PCR reaction input. Concentrations of HDRTs are determined by nanodrop using a 1:20 dilution.

Single-stranded DNA HDRT production by exonuclease digestion: To produce long ssDNA as HDR templates, the DNA of interest was amplified via PCR using one regular, non-modified PCR primer and a second phosphorylated PCR primer. The DNA strand that will be amplified using the phosphorylated primer will be the strand that will be degraded using this method. This makes it possible to prepare either a single-stranded sense or single-stranded antisense DNA using the respective phosphorylated PCR primer. To produce the ssDNA strand of interest, the phosphorylated strand of the PCR product was degraded by treatment with two enzymes, Strandase Mix A and Strandase Mix B, for 5 min (per 1 kb) at 37 °C, respectively. Enzymes were deactivated by a 5 min incubation at 80 °C. The resulting ssDNA HDR templates are purified and eluted in H₂O. A more detailed protocol for the Guide-it Long ssDNA Production System (Takara Bio, 632644) can be found at the manufacturer's website.

### Electroporation Method

Wash T cells to be electroporated (X 3 times) using Opti-MEM I: Reduced serum medium (Catalog no. 31985, Gibco, Grand Island, NY, USA). Centrifuge cells at 300xg for 5 minutes at 4 °C.

Carefully discard supernatant and resuspend cell pellet in fresh Opti-MEM media at 1×108 cells/ml. For each electroporation, aliquot 1×107 cells in a 100 μl of Opti-MEM. Keep cells on ice until use.

Pre-configure the electroporator by setting the voltage to 500V and time to 1000μ-seconds.

Prewarm R10 media to 37 °C and add 10 ml of the media to a T25 flask.

Place the cells in 100 μL into a 2 mm cuvette.

Add 10 uL of RNP complex plus 2 μL HDRT with the 100 μl aliquot of cells. Uniformly mix by gentle pipetting.

Place the cuvette into the electroporator cassette, tighten the electrodes around the metal plates of the cuvette and initiate the electric pulse.

Immediately transfer the contents of the cuvette into the T25 flask containing R10.

Rinse the cuvette once with fresh R10 to maximize recovery of electroporated cells.

Place the cells in a 37 °C. CO2 incubator until further use.

After 25 hrs, positively electroporated cells can be sorted by using the fluorescent label on the tracRNA (550 nm), after 24 hrs. To verify expression of the CER on the cell surface FACS analysis using anti-FSH Abs can be used, or the expression of FSH can be tested by Q-PCR using the primers from Example 1.

Electropotared, CRISPRed T cells are then expanded against irradiated K562 cells transduced with human FSHR (1:10 ratio), in R10 media with II-2 (300 UI/ml).

### HDRT Sequence (PERK+P2A+tg)

5′-arm:

(SEQ ID NO: 13)
CAGCACTTTAGATGGGAGAATTGCTGCCTTGGATCCTGAAAATCATGGT
AAAAAGCAGTGGGATTTGGATGTGGGATCCGGTTCCTTGGTGTCATCCA
GCCTTAGCAAACCAGAGGTATTTGGGAATAAGATGATCATTCCTTCCCT
GGATGGAGCCCTCTTCCAGTGGGACCAAGACCGTGAAAGCATGGAAACA
GTTCCTTTCACAGTTGAATCACTTCTTGAATCTTCTTATAAATTTGGAG
ATGATGTTGTTTTGGTTGGAGGAAAATCTCTGACTACATATGGACTCAG
TGCATA.

g+P2A (Inserted at Place of Crispr Excision (AUA\UAG):

(SEQ ID NO: 14)
tggccacgaacttctccctgttaaagCaagcaggagacgtggaagaaaac
cccggtccc.

### FSHCER:

(SEQ ID NO: 10)
ATGAAGACCCTGCAGTTCTTCTTCCTGTTCTGCTGCTGGAAGGCCATCTG
CTGCAACAGCTGCGAGCTGACCAACATCACAATCGCCATCGAGAAAGAGG
AATGCCGGTTCTGCATCAGCATCAACACCACTTGGTGCGCCGGCTACTGC
TACACCCGGGACCTGGTGTACAAGGACCCCGCCAGACCCAAGATCCAGAA
AACCTGCACCTTCAAAGAACTGGTGTACGAGACAGTGCGGGTGCCCGGAT
GTGCCCACCATGCCGATAGCCTGTACACCTACCCTGTGGCCACCCAGTGT
CACTGCGGCAAGTGCGATAGCGACAGCACCGATTGCACCGTGCGGGGACT
GGGCCCTAGCTACTGTAGCTTCGGCGAGATGAAGGAAGGCGGCGGATCTG
GCGGAGGAAGCGGAGGGGGATCTGGGGGCGGAGCACCTGATGTGCAGGAT
TGCCCTGAGTGCACCCTGCAGGAAAACCCATTCTTCAGCCAGCCTGGCGC
CCCTATCCTGCAGTGCATGGGCTGCTGCTTCAGCAGAGCCTACCCCACCC
CCCTGCGGAGCAAGAAAACCATGCTGGTGCAGAAAAACGTGACCAGCGAG
AGCACCTGTTGCGTGGCCAAGAGCTACAACAGAGTGACCGTGATGGGCGG
CTTCAAGGTGGAAAACCACACCGCCTGCCACTGCAGCACATGCTACTACC
ACAAGAGCGCTAGCACCACCACCCCTGCCCCTAGACCTCCAACACCCGCC
CCTACAATCGCCTCCCAGCCTCTGTCTCTGAGGCCCGAGGCTTGTAGACC
AGCTGCTGGCGGAGCCGTGCACACCAGAGGACTGGATTTCGCCTGCGACA
TCTACATCTGGGCCCCTCTGGCCGGCACATGTGGCGTGCTGCTGCTGAGC
CTCGTGATCACCCTGTACTGCAAGCGGGGCAGAAAGAAGCTGCTGTACAT
CTTCAAGCAGCCCTTCATGCGGCCCGTGCAGACCACCCAGGAAGAGGACG
GCTGCTCCTGCAGATTCCCCGAAGAGGAAGAGGGGGGCTGCGAACTGAGA
GTGAAGTTCAGCAGAAGCGCCGACGCCCCTGCCTACAAGCAGGGCCAGAA
CCAGCTGTACAACGAGCTGAACCTGGGCAGACGGGAAGAGTACGACGTGC
TGGACAAGCGGAGAGGCAGGGACCCTGAGATGGGCGGAAAGCCCAGACGG
AAGAACCCCCAGGAAGGCCTGTATAACGAACTGCAGAAAGACAAGATGGC
CGAGGCCTACAGCGAGATCGGAATGAAGGGCGAGCGGAGAAGAGGCAAGG
GCCACGATGGCCTGTACCAGGGCCTGAGCACCGCCACCAAGGACACCTAT
GACGCCCTGCACATGCAGGCCCTGCCCCCCAGATAA.

3′-arm:

(SEQ ID NO: 15)
TAGTGGAAAGGTGAGATGGACTCAGTGCATATAGTGGAAAGGTAAGTGAA
AATGCTGAATTTACTTTGGGGAAATCAGAGTAAATTAGGGTAGAAAAAGT
AATTTATTAAACTACACTTATTATTAGTTGAGTTTTATTGTAATTTTCCC
CTGAGGTTGTCATTTGTTTTAATAAGAGAACTGTGAGGTAGGAAGGGGAA
ACTAATAACAGAATAAATGGCAGAGCCAGGAATAGCAGGAGGAAGAGAAT
TCATAAATATGGTCTACTGTGTCTCAGGTGTTTTTTTTTTT.

### HDRT Sequence:

(SEQ ID NO: 16)
CAGCACTTTAGATGGGAGAATTGCTGCCTTGGATCCTGAAAATCATGGTA
AAAAGCAGTGGGATTTGGATGTGGGATCCGGTTCCTTGGTGTCATCCAGC
CTTAGCAAACCAGAGGTATTTGGGAATAAGATGATCATTCCTTCCCTGGA
TGGAGCCCTCTTCCAGTGGGACCAAGACCGTGAAAGCATGGAAACAGTTC
CTTTCACAGTTGAATCACTTCTTGAATCTTCTTATAAATTTGGAGATGAT
GTTGTTTTGGTTGGAGGAAAATCTCTGACTACATATGGACTCAGTGCATA
tggccacgaacttctccctgttaaagCaagcaggagacgtggaagaaaac
cccggtcccATGAAGACCCTGCAGTTCTTCTTCCTGTTCTGCTGCTGGAA
GGCCATCTGCTGCAACAGCTGCGAGCTGACCAACATCACAATCGCCATCG
AGAAAGAGGAATGCCGGTTCTGCATCAGCATCAACACCACTTGGTGCGCC
GGCTACTGCTACACCCGGGACCTGGTGTACAAGGACCCCGCCAGACCCAA
GATCCAGAAAACCTGCACCTTCAAAGAACTGGTGTACGAGACAGTGCGGG
TGCCCGGATGTGCCCACCATGCCGATAGCCTGTACACCTACCCTGTGGCC
ACCCAGTGTCACTGCGGCAAGTGCGATAGCGACAGCACCGATTGCACCGT
GCGGGGACTGGGCCCTAGCTACTGTAGCTTCGGCGAGATGAAGGAAGGCG
GCGGATCTGGCGGAGGAAGCGGAGGGGGATCTGGGGGCGGAGCACCTGAT
GTGCAGGATTGCCCTGAGTGCACCCTGCAGGAAAACCCATTCTTCAGCCA
GCCTGGCGCCCCTATCCTGCAGTGCATGGGCTGCTGCTTCAGCAGAGCCT
ACCCCACCCCCCTGCGGAGCAAGAAAACCATGCTGGTGCAGAAAAACGTG
ACCAGCGAGAGCACCTGTTGCGTGGCCAAGAGCTACAACAGAGTGACCGT
GATGGGCGGCTTCAAGGTGGAAAACCACACCGCCTGCCACTGCAGCACAT
GCTACTACCACAAGAGCGCTAGCACCACCACCCCTGCCCCTAGACCTCCA
ACACCCGCCCCTACAATCGCCTCCCAGCCTCTGTCTCTGAGGCCCGAGGC
TTGTAGACCAGCTGCTGGCGGAGCCGTGCACACCAGAGGACTGGATTTCG
CCTGCGACATCTACATCTGGGCCCCTCTGGCCGGCACATGTGGCGTGCTG
CTGCTGAGCCTCGTGATCACCCTGTACTGCAAGCGGGGCAGAAAGAAGCT
GCTGTACATCTTCAAGCAGCCCTTCATGCGGCCCGTGCAGACCACCCAGG
AAGAGGACGGCTGCTCCTGCAGATTCCCCGAAGAGGAAGAGGGGGGCTGC
GAACTGAGAGTGAAGTTCAGCAGAAGCGCCGACGCCCCTGCCTACAAGCA
GGGCCAGAACCAGCTGTACAACGAGCTGAACCTGGGCAGACGGGAAGAGT
ACGACGTGCTGGACAAGCGGAGAGGCAGGGACCCTGAGATGGGCGGAAAG
CCCAGACGGAAGAACCCCCAGGAAGGCCTGTATAACGAACTGCAGAAAGA
CAAGATGGCCGAGGCCTACAGCGAGATCGGAATGAAGGGCGAGCGGAGAA
GAGGCAAGGGCCACGATGGCCTGTACCAGGGCCTGAGCACCGCCACCAAG
GACACCTATGACGCCCTGCACATGCAGGCCCTGCCCCCCAGATAATAGTG
GAAAGGTGAGATGGACTCAGTGCATATAGTGGAAAGGTAAGTGAAAATGC
TGAATTTACTTTGGGGAAATCAGAGTAAATTAGGGTAGAAAAAGTAATTT
ATTAAACTACACTTATTATTAGTTGAGTTTTATTGTAATTTTCCCCTGAG
GTTGTCATTTGTTTTAATAAGAGAACTGTGAGGTAGGAAGGGGAAACTAA
TAACAGAATAAATGGCAGAGCCAGGAATAGCAGGAGGAAGAGAATTCATA
AATATGGTCTACTGTGTCTCAGGTGTTTTTTTTTT.

### Example 3: Successful Integration of an HLA-Independent T Cell (HIT) Receptor into the PERK Locus

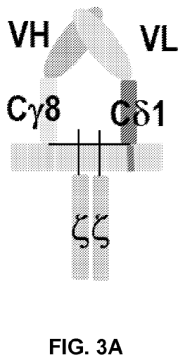

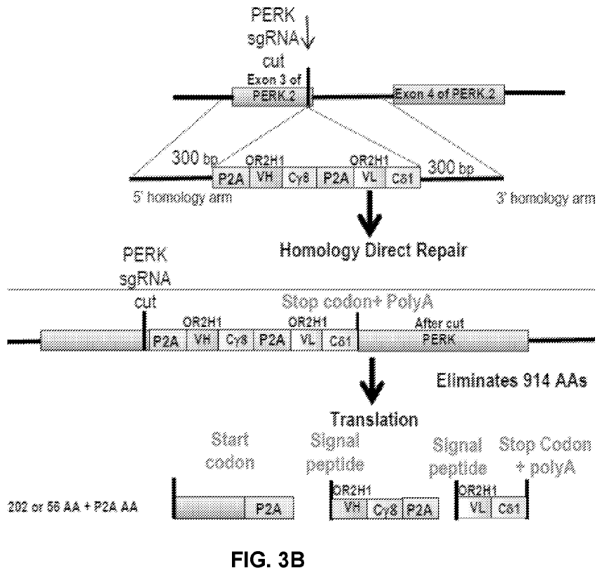

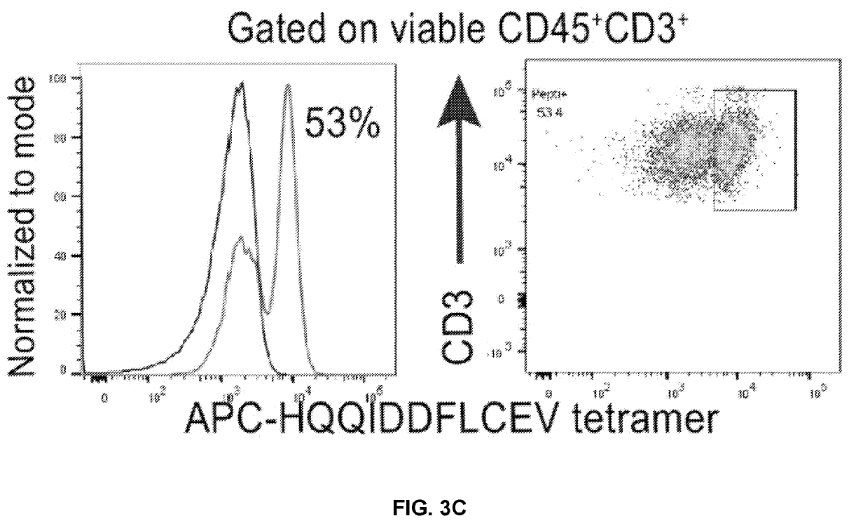

Successful integration of an HLA-independent T cell (HIT) receptor into the PERK locus. Our collaborative work demonstrated that PERK-CHOP and IRE-1α-XBP1 blunt metabolic fitness of ovarian cancer-reactive T cells (Song, M., et al. Nature 2018 562:423-428; Cao, Y., et al. Nat Commun 2019 10:1280). On the other hand, utilizing the same VH/VL chains of the targeting motif of a CD19 CAR, the Sadelain group recently demonstrated the feasibility of integrating into the TRAC locus a T cell receptor-CD3 complex (termed HIT) in human peripheral blood T cells4. HIT T cells show higher antigen sensitivity than conventional CARs, making this approach ideal for targets expressed at low levels. Adapting the same protocol, we integrated a Vγ8Vγ1 TCR instead of an αβ TCR into αβ T cells, using the PERK locus instead of TCRa. We choose a γδ TCR instead of an αβ TCR because: 1) Signaling by γδ TCRs results in stronger proliferative and effector responses, compared to αβ TCRs (Hayes, S.M. & Love, P.E. Immunity 2002 16:827-838); 2) γδ TCR-based HIT could preserve BTNL9-dependent T cell activation at tumor beds; and 3) preserving the endogenous αβ TCR reactivity. Integrating the HIT construct into the PERK locus takes advantage of the constitutive expression of PERK, while ablating this immunosuppressive arm of the UPR response (Song, M., et al. Nature 2018 562:423-428; Cao, Y., et al. Nat Commun 2019 10:1280). For proof of concept, we integrated our OR2H1 CAR1, which is being developed for clinical testing and targets ~69% of intra-hepatic cholangiocarcinomas, ~38% of prostate cancers and other tumors, but only testis among healthy tissues1. A scheme of the approach and proof of γδ TCR expression is show in FIG. 3.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A chimeric cell expressing a chimeric receptor,
wherein the chimeric receptor is encoded by a transgene, and
wherein the transgene is inserted in the genome of the cell at a location that disrupts expression or activity of an endogenous X-box binding protein 1 (XBP1) and/or protein kinase R (PKR)-like endoplasmic reticulum kinase (PERK).

2. The chimeric cell of claim 1, wherein the chimeric receptor is a chimeric antigen receptor (CAR) polypeptide.

3. The chimeric cell of claim 2, wherein the chimeric receptor comprises two subunits of a follicule-stimulating hormone (FSH).

4. The chimeric cell of claim 3, wherein the chimeric receptor comprises the amino acid sequence SEQ ID NO:1, or a variant thereof having at least 90% sequence identity to SEQ ID NO:1

5. The chimeric cell of claim 1, wherein the cell is selected from the group consisting of an αβT cell, γδT cell, a Natural Killer (NK) cells, a Natural Killer T (NKT) cell, a B cell, an innate lymphoid cell (ILC), a cytokine induced killer (CIK) cell, a cytotoxic T lymphocyte (CTL), a lymphokine activated killer (LAK) cell, a regulatory T cell, or any combination thereof.

6. The chimeric cell of claim 3, wherein the cell exhibits an anti-tumor immunity when the antigen binding domain of the chimeric receptor binds to an FSH-receptor positive ovarian tumor.

7. A method of providing an anti-cancer immunity in a subject, comprising administering to the subject an effective amount of the chimeric cell of claim 1, thereby providing an anti-tumor immunity in the subject.

8. The method of claim 7, wherein the chimeric receptor comprises two subunits of a follicule-stimulating hormone (FSH), and wherein the subject has an FSH-receptor positive ovarian tumor.

9. The method of claim 7, further comprising administering to the subject a checkpoint inhibitor.

10. The method of claim 9, wherein the checkpoint inhibitor comprises an anti-PD-1 antibody, anti-PD-L1 antibody, anti-CTLA-4 antibody, or a combination thereof.
